SEQUENCE LISTING (15 sequences)

SEQ ID NO: 1 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 1368 aa, synthetic construct; identical to SEQ ID NO: 1
  REGION 1..1368: note = S. pyogenes nCas9 Protein Sequence having the D10A mutation

SEQ ID NO: 3 - protein, 1368 aa, synthetic construct
  REGION 1..1368: note = S. pyogenes nCas9 Protein Sequence having the H840A mutation
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 4 - protein, 167 aa, Staphylococcus aureus
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 5 - protein, 5 aa, synthetic construct
  REGION 1..5: note = linker
GGGGS

SEQ ID NO: 6 - protein, 5 aa, synthetic construct
  REGION 1..5: note = linker
EAAAK

SEQ ID NO: 7 - protein, 16 aa, synthetic construct
  REGION 1..16: note = Linker
SGSETPGTSESATPES

SEQ ID NO: 8 - protein, 1877 aa, synthetic construct
  REGION 1..1877: note = SpRY-ABE8e
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGDSGGSKRTADGSEFEPKKKRKVGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGGSPKKKRKV

SEQ ID NO: 9 - DNA, 20 bp, Homo sapiens
  source 1..20: mol_type = unassigned DNA
ttagtctattttcccaccct

SEQ ID NO: 10 - DNA, 20 bp, Homo sapiens
  source 1..20: mol_type = unassigned DNA
attagtctattttcccaccc

SEQ ID NO: 11 - DNA, 20 bp, Homo sapiens
  source 1..20: mol_type = unassigned DNA
tattagtctattttcccacc

SEQ ID NO: 12 - DNA, 20 bp, Homo sapiens
  source 1..20: mol_type = unassigned DNA
ctattagtctattttcccac

SEQ ID NO: 13 - DNA, 20 bp, Homo sapiens
  source 1..20: mol_type = unassigned DNA
cctattagtctattttccca

SEQ ID NO: 14 - DNA, 20 bp, Homo sapiens
  source 1..20: mol_type = unassigned DNA
gcctattagtctattttccc

SEQ ID NO: 15 - DNA, 39 bp, Homo sapiens
  source 1..39: mol_type = unassigned DNA
tctctctgcctattagtctattttcccacccttaggctg

(19) United States
(12) Patent Application Publication
(10) Pub. No.: US 2025/0161492 A1
(43) Pub. Date: May 22, 2025

(54) BASE EDITING APPROACHES FOR THE TREATMENT OF BETA-THALASSEMIA

(71) Applicants: INSTITUT NATIONAL DE LA SANTÉ ET DE LA RECHERCHE MÉDICALE, Paris (FR); FONDATION IMAGINE, Paris (FR); ASSISTANCE PUBLIQUE-HÔPITAUX DE PARIS (APHP), Paris (FR); UNIVERSITÉ PARIS CITÉ, Paris (FR)

(72) Inventors: Annarita MICCIO, Paris (FR); Giulia HARDOUIN, Paris (FR)

(21) Appl. No.: 18/725,334

(22) PCT Filed: Jan. 24, 2023
(86) PCT No.: PCT/EP2023/051599

§ 371 Date: Jun. 28, 2024

(30) Foreign Application Priority Data
Jan. 25, 2022 (EP) 22305075.8

(51) Int. Cl. A61K 48/00; C12N 9/22; C12N 9/78; C12N 15/11

(52) U.S. Cl. CPC A61K 48/0058; C12N 9/22; C12N 9/78; C12N 15/11; C12Y 305/04004; C12N 2310/20

(57) ABSTRACT

IVS1-110 (G>A) is one of the most common β-thalassemic mutations in the Middle East and Mediterranean area, representing >75% and >40% of β-thalassemic mutations in Cyprus and Greece, respectively. This point mutation is classified as a severe β⁺ mutation, and homozygous patients or compound heterozygotes harboring this mutation in combination with a β⁰ mutation have a clinical phenotype similar to β⁰/β⁰ patients. Here, the inventors exploited adenine base-editors (ABEs) to correct the IVS1-110 (G>A) mutation in HSPCs from β-thalassemia patients and demonstrated the potential of this strategy to correct the pathological phenotype C observed during erythroid differentiation. In particular the inventors demonstrated that reverting the IVS1-110 (G>A) mutation using base editing corrected the β-thalassemic cell phenotype in vitro and in vivo. The present invention thus relates to base editing approaches for the treatment of β-thalassemia including sickle β-thalassemia.

### FIELD OF THE INVENTION

The present invention is in the field of medicine, in particular haematology.

### BACKGROUND OF THE INVENTION

β-globin chains together with α-globin chains compose the adult hemoglobin (HbA) tetramer. β-thalassemia is a monogenic recessive disease caused by a variety of mutations affecting the synthesis of the adult hemoglobin 0-chains. It is a highly prevalent hemoglobinopathy with 68,000 affected children annually worldwide¹. Patients were originally concentrated in Asia, India and the Mediterranean region but due to recent population movements, β-thalassemia is now widely spread in Europe and North America¹. Point mutations or deletions in the β-globin gene (HBB) locus reduce (β⁺) or abolish (β⁰) the production of β-globin chains. The imbalance between α- and β-globin production, leads to the precipitation of uncoupled α-globins, which causes erythroid cell death, ineffective erythropoiesis and severe anemia². Depending on their genotype, the clinical phenotype can vary from mild to severe anemia, known as β-thalassemia major (typically associated with a β⁰/β⁰ genotype). In the most severe cases, patients are transfusion-dependent and require an iron-chelation therapy to alleviate iron overload due to chronic transfusions.

IVS1-110 (G>A) is one of the most common β-thalassemic mutations in the Middle East and Mediterranean area, representing >75% and >40% of β-thalassemic mutations in Cyprus and Greece, respectively³⁻⁵. This point mutation is located in the first intron of HBB and leads to the formation of a de novo splice acceptor site causing abnormal splicing of 90% of the β-globin transcripts⁶. As β-globin expression is strongly down-regulated, IVS1-110 (G>A) is classified as a severe β⁺ mutation, and homozygous patients or compound heterozygotes harboring this mutation in combination with a 1° mutation have a clinical phenotype similar to β⁰/β⁰ patients⁷.

The only definitive cure for β-thalassemia patients is the transplantation of allogeneic hematopoietic stem cells (HSCs), but this treatment is limited by the availability of HLA-compatible donors. Gene therapy approaches based on the transplantation of autologous, genetically modified HSCs have been investigated as a treatment option for patients lacking a compatible donor⁸.

Genome editing technology has been exploited to develop therapeutic approaches for β-hemoglobinopathies. These approaches use designer nucleases, such as the CRISPR/Cas9 nuclease system that induces DNA double-strand breaks (DSBs) via a single guide RNA (gRNA) complementary to a specific genomic target. The DSB can be repaired via homologous-directed repair (HDR), by providing a donor DNA template containing the wild type sequence, allowing direct gene correction of the mutation. However, HDR-mediated gene correction is poorly efficient in HSCs⁹. Nuclease-based strategies based on the disruption of the cryptic splice site generated by the IVS1-110 (G>A) mutation through InDel (insertion/deletion) formation after non-homologous end-joining-mediated repair of DSBs were effective in restoring correct Hb expression^{10,11}. However, only a fraction of InDels abolished the aberrant splice acceptor site¹¹. Furthermore, HSCs are highly sensitive to DNA double-strand breaks (DSBs)¹²—especially in cases of multiple on-targets or concomitant on-target and off-target events. Even when highly specific gRNAs are used, Cas9-gRNA treatment of human hematopoietic/stem progenitor cells (HSPCs) induces a DNA damage response that can lead to apoptosis^{13,14}. CRISPR-Cas9 can cause p53-dependent cell toxicity and cell cycle arrest, resulting in the negative selection of cells with a functional p53 pathway¹⁵. Furthermore, the generation of several on-target DSBs, simultaneous on-target and off-target DSBs, or even a single on-target DSB is associated with a risk of deletion, inversion and translocation¹⁶⁻¹⁹.

Base editing, a new CRISPR/Cas9-derived genome editing tool, allows precise DNA repair in bonafide HSCs²⁰ without the occurrence of DSBs. Adenine base editors (ABE) and cytosine base editors (CBE) contain a Cas9 nickase and a deaminase, and permit the insertion of A>G and C>T mutations, respectively²¹. Base editing has been exploited to correct a β-thalassemia-causing mutation in the HBB promoter^{20,22}.

### SUMMARY OF THE INVENTION

The present invention is defined by the claims. In particular, the present invention relates to base editing approaches for the treatment of β-thalassemia.

### DETAILED DESCRIPTION OF THE INVENTION

Here, the inventors exploited adenine base-editors (ABEs) to correct the IVS1-110 (G>A) mutation in HSPCs from β-thalassemia patients and demonstrated the potential of this strategy to correct the pathological phenotype observed during erythroid differentiation.

### Definitions

As used herein, the term “β-thalassemia” refers to a hemoglobinopathy that results from an altered ratio of α-globin to β-like globin polypeptide chains resulting in the underproduction of normal hemoglobin tetrameric proteins and the precipitation of free, unpaired α-globin chains.

As used herein, the term “sickle β-thalassemia” refers to a particular form of β-thalassemia wherein the patient has a mutation in each copy of their HBB gene: one that causes red blood cells to form a “sickle” or crescent shape and a second that is associated with beta thalassemia, a blood disorder that reduces the production of hemoglobin. Clinical manifestations depend on the amount of residual beta globin chains production, and are similar to sickle cell disease, including anemia, vascular occlusion and its complications, acute episodes of pain, acute chest syndrome, pulmonary hypertension, sepsis, ischemic brain injury, splenic sequestration crisis and splenomegaly.

As used herein, the term “hematopoietic stem cell” or “HSC” refers to blood cells that have the capacity to self-renew and to differentiate into precursors of blood cells. These precursor cells are immature blood cells that cannot self-renew and must differentiate into mature blood cells. Hematopoietic stem progenitor cells display a number of phenotypes, such as Lin-CD34+CD38−CD90+CD45RA−, Lin-CD34+CD38−CD90−CD45RA−, Lin-CD34+CD38+IL-3aloCD45RA−, and Lin-CD34+CD38+CD10+(Daley et al., Focus 18:62-67, 1996; Pimentel, E., Ed., Handbook of Growth Factors Vol. III: Hematopoietic Growth Factors and Cytokines, pp. 1-2, CRC Press, Boca Raton, Fla., 1994). Within the bone marrow microenvironment, the stem cells self-renew and maintain continuous production of hematopoietic stem cells that give rise to all mature blood cells throughout life. In some embodiments, the hematopoietic progenitor cells or hematopoietic stem cells are isolated form peripheral blood cells.

As used herein, the term “peripheral blood cells” refer to the cellular components of blood, including red blood cells, white blood cells, and platelets, which are found within the circulating pool of blood. In some embodiments, the eukaryotic cell is a bone marrow derived stem cell.

As used herein the term “bone marrow-derived stem cells” refers to stem cells found in the bone marrow. Stem cells may reside in the bone marrow, either as an adherent stromal cell type that possess pluripotent capabilities, or as cells that express CD34 or CD45 cell-surface protein, which identifies hematopoietic stem cells able to differentiate into blood cells.

As used herein, the term “mobilization” or “stem cell mobilization” refers to a process involving the recruitment of stem cells from their tissue or organ of residence to peripheral blood following treatment with a mobilization agent. This process mimics the enhancement of the physiological release of stem cells from tissues or organs in response to stress signals during injury and inflammation. The mechanism of the mobilization process depends on the type of mobilization agent administered. Some mobilization agents act as agonists or antagonists that prevent the attachment of stem cells to cells or tissues of their microenvironment. Other mobilization agents induce the release of proteases that cleave the adhesion molecules or support structures between stem cells and their sites of attachment.

As used herein, the term “mobilization agent” refers to a wide range of molecules that act to enhance the mobilization of stem cells from their tissue or organ of residence, e.g., bone marrow (e.g., CD34+ stem cells) and spleen (e.g., Hox11+ stem cells), into peripheral blood. Mobilization agents include chemotherapeutic drugs, e.g., cyclophosphamide and cisplatin; cytokines, and chemokines, e.g., granulocyte colony-stimulating factor (G-CSF), granulocyte-macrophage colony-stimulating factor (GM-CSF), stem cell factor (SCF), Fms-related tyrosine kinase 3 (flt-3) ligand, stromal cell-derived factor 1 (SDF-1); agonists of the chemokine (C—C motif) receptor 1 (CCR1), such as chemokine (C—C motif) ligand 3 (CCL3, also known as macrophage inflammatory protein-1α (Mip-1α)); agonists of the chemokine (C—X—C motif) receptor 1 (CXCR1) and 2 (CXCR2), such as chemokine (C—X—C motif) ligand 2 (CXCL2) (also known as growth-related oncogene protein-β (Gro-β)), and CXCL8 (also known as interleukin-8 (IL-8)); agonists of CXCR4, such as CTCE-02142, and Met-SDF-1,; Very Late Antigen (VLA)-4 inhibitors; antagonists of CXCR4, such as TG-0054, plerixafor (also known as AMD3100), and AMD3465, or any combination of the previous agents. A mobilization agent increases the number of stem cells in peripheral blood, thus allowing for a more accessible source of stem cells for use in transplantation, organ repair or regeneration, or treatment of disease.

As used herein, the term “isolated cell” refers to a cell that has been removed from an organism in which it was originally found, or a descendant of such a cell. Optionally the eukaryotic cell has been cultured in vitro, e.g., in the presence of other cells. Optionally the eukaryotic cell is later introduced into a second organism or reintroduced into the organism from which it (or the cell from which it is descended) was isolated. As used herein, the term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched.

As used herein, the terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, pegylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the term “nucleic acid molecule” or “polynucleotide” refers to a DNA molecule (for example, but not limited to, a cDNA or genomic DNA). The nucleic acid molecule can be single-stranded or double-stranded.

As used herein, the term “isolated” when referring to nucleic acid molecules or polypeptides means that the nucleic acid molecule or the polypeptide is substantially free from at least one other component with which it is associated or found together in nature.

As used herein, the term “complementarity” refers to the ability of a nucleic acid to form hydrogen bond(s) with another nucleic acid sequence by either traditional Watson-Crick base-pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, the term “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

As used herein, the term “hybridization” or “hybridizing” refers to a process where completely or partially complementary nucleic acid strands come together under specified hybridization conditions to form a double-stranded structure or region in which the two constituent strands are joined by hydrogen bonds. Although hydrogen bonds typically form between adenine and thymine or uracil (A and T or U) or cytosine and guanine (C and G), other base pairs may form (e.g., Adams et al., The Biochemistry of the Nucleic Acids, 11th ed., 1992).

As used herein the term “wild type” is a term of the art understood by skilled persons and means the typical form of an organism, strain, gene or characteristic as it occurs in nature as distinguished from mutant or variant forms.

As used herein, the term “mutation” has its general meaning in the art and refers to a substitution, deletion or insertion. The term “substitution” means that a specific amino acid residue at a specific position is removed and another amino acid residue is inserted into the same position. The term “deletion” means that a specific amino acid residue is removed. The term “insertion” means that one or more amino acid residues are inserted before or after a specific amino acid residue.

As used herein, the term “mutagenesis” refers to the introduction of mutations into a polynucleotide sequence. According to the present invention mutations are introduced into a target DNA molecule.

As used herein, the term “variant” refers to a first composition (e.g., a first molecule), that is related to a second composition (e.g., a second molecule, also termed a “parent” molecule). The variant molecule can be derived from, isolated from, based on or homologous to the parent molecule. A variant molecule can have entire sequence identity with the original parent molecule, or alternatively, can have less than 100% sequence identity with the parent molecule. For example, a variant of a sequence can be a second sequence that is at least 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100% identical in sequence compare to the original sequence.

As used herein, the “percent identity” between the two sequences is a function of the number of identical positions shared by the sequences (i.e., % identity=number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the Needleman and Wunsch algorithm (Needleman, Saul B. & Wunsch, Christian D. (1970). “A general method applicable to the search for similarities in the amino acid sequence of two proteins”. Journal of Molecular Biology. 48 (3): 443-53.). The percent identity between two nucleotide or amino acid sequences may also be determined using for example algorithms such as EMBOSS Needle (pair wise alignment; available at www.ebi.ac.uk). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification. According to the invention a first amino acid sequence having at least 90% of identity with a second amino acid sequence means that the first sequence has 90; 91; 92; 93; 94; 95; 96; 97; 98; 99 or 100% of identity with the second amino acid sequence.

As used herein, the term “alpha globin” or “α-globin” has its general meaning in the art and refers to protein that is encoded in human by the HBA1 and HBA2 genes. The human alpha globin gene cluster located on chromosome 16 spans about 30 kb and includes seven loci: 5′-zeta-pseudozeta-mu-pseudoalpha-1-alpha-2-alpha-1-theta-3′. The alpha-2 (HBA2) and alpha-1 (HBA1) coding sequences are identical. These genes differ slightly over the 5′ untranslated regions and the introns, but they differ significantly over the 3′ untranslated regions. The ENSEMBL IDs (i.e. the gene identifier number from the Ensembl Genome Browser database) for HBA1 and HBA2 are ENSG00000206172 and ENSG00000188536 respectively.

As used herein, the term “beta globin” or “β-globin” has its general meaning in the art and refers to a globin protein, which along with alpha globin (HBA), makes up the most common form of haemoglobin (Hb) in adult humans. Normal adult human Hb is a heterotetramer consisting of two alpha chains and two beta chains. HBB is encoded by the HBB gene on human chromosome 11. It is 146 amino acids long and has a molecular weight of 15,867 Da.

As used herein, the term “IVS1-110 (G>A) mutation” or “HBB:c.93-21G>A” has its general meaning in the art and refers to one of the most common β-thalassemic mutations in the Middle East and Mediterranean area, representing >75% and >40% of β-thalassemic mutations in Cyprus and Greece, respectively³⁻⁵. This point mutation is located in the first intron of HBB and leads to the formation of a de novo splice acceptor site causing abnormal splicing of 90% of the β-globin transcripts⁶.

As used herein, the term “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. Any method known in the art can be used to measure the expression of the gene (e. g. HPLC analysis of protein and RT-qPCR analysis of mRNA.) Typically, said methods are described in the EXAMPLE.

As used herein, the expression “restoring the normal expression of β-globin” indicates that the expression of β-globin is restored to at approximately the same level as for an eukaryotic cell that does not carry the IVS1-110 (G>A) mutation (i.e. an eukaryotic carrying the wild type HBB gene).

As used herein, the term “derived from” refers to a process whereby a first component (e.g., a first molecule), or information from that first component, is used to isolate, derive or make a different second component (e.g., a second molecule that is different from the first).

As used herein, the term “fusion polypeptide” or “fusion protein” means a protein created by joining two or more polypeptide sequences together. The fusion polypeptides encompassed in this invention include translation products of a chimeric gene construct that joins the nucleic acid sequences encoding a first polypeptide, e.g., an RNA-binding domain, with the nucleic acid sequence encoding a second polypeptide, e.g., an effector domain, to form a single open-reading frame. In other words, a “fusion polypeptide” or “fusion protein” is a recombinant protein of two or more proteins which are joined by a peptide bond or via several peptides. The fusion protein may also comprise a peptide linker between the two domains.

As used herein, the term “linker” refers to any means, entity or moiety used to join two or more entities. A linker can be a covalent linker or a non-covalent linker. Examples of covalent linkers include covalent bonds or a linker moiety covalently attached to one or more of the proteins or domains to be linked. The linker can also be a non-covalent bond, e.g., an organometallic bond through a metal center such as platinum atom. For covalent linkages, various functionalities can be used, such as amide groups, including carbonic acid derivatives, ethers, esters, including organic and inorganic esters, amino, urethane, urea and the like. To provide for linking, the domains can be modified by oxidation, hydroxylation, substitution, reduction etc. to provide a site for coupling. Methods for conjugation are well known by persons skilled in the art and are encompassed for use in the present invention. Linker moieties include, but are not limited to, chemical linker moieties, or for example a peptide linker moiety (a linker sequence). It will be appreciated that modification which do not significantly decrease the function of the RNA-binding domain and effector domain are preferred.

As used herein, the “linked” as used herein refers to the attachment of two or more entities to form one entity. A conjugate encompasses both peptide-small molecule conjugates as well as peptide-protein/peptide conjugates.

As used herein, the term “base-editor” refers to fusion protein comprising a defective CRISPR/Cas nuclease linked to a deaminase polypeptide. Two classes of base-editors-“cytosine base-editors” (CBEs) and “adenine base-editors” (ABEs)—can be used to generate single base pair edits without double stranded breaks. Typically, base-editor are created by fusing the defective CRISPR/Cas nuclease to a deaminase.

As used herein, the term “deaminase” refers to an enzyme that catalyses a deamination reaction. The term “deamination”, as used herein, refers to the removal of an amine group from one molecule. In some embodiments, the deaminase is a “cytidine deaminase”, catalysing the hydrolytic deamination of cytidine or deoxycytidine to uracil or deoxyuracil, respectively. In some embodiments, the deaminase is an “adenosine deaminase”, catalysing the hydrolytic deamination of adenosine to inosine, which is treated like guanosine by the cell, creating an A to G (or T to C) change.

As used herein, the term “nuclease” includes a protein (i.e. an enzyme) that induces a break in a nucleic acid sequence, e.g., a single or a double strand break in a double-stranded DNA sequence.

As used herein, the term “CRISPR/Cas nuclease” has its general meaning in the art and refers to segments of prokaryotic DNA containing clustered regularly interspaced short palindromic repeats (CRISPR) and associated nucleases encoded by Cas genes. In bacteria the CRISPR/Cas loci encode RNA-guided adaptive immune systems against mobile genetic elements (viruses, transposable elements and conjugative plasmids). Three types of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR/Cas nucleases Cas9 and Cpf1 belong to the type II and type V CRISPR/Cas system and have strong endonuclease activity to cut target DNA. Cas9 is guided by a mature crRNA that contains about 20 nucleotides of unique target sequence (called spacer) and a trans-activating small RNA (tracrRNA) that also serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG for S. Pyogenes Cas9) protospacer adjacent motif (PAM) to specify the cut site (the 3^{rd} or the 4^{th} nucleotide upstream from PAM).

As used herein, the term “Cas9” or “Cas9 nuclease” refers to an RNA-guided nuclease comprising a Cas9 protein, or a fragment thereof (e.g., a protein comprising an active or inactive DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, the term “Cas9” refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisl (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria. meningitidis (NCBI Ref: YP_002342100.1). Typically the Cas9 nuclease comprises the amino acid sequence as set forth in SEQ ID NO: 1.

SEQ ID NO: 1: Cas9 sequence
MDKKYSIGLAIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEENPINASGVD
AKAILSARLSKSRRLENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNGSIPHQI
HLGELHAILRRQEDFYPFLKDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDFLDNEENEDILEDIVL
TLTLFEDREMIEERLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDELVKVMGRHKPENI
VIEMARENQTTQKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQLLNAKLITQRKF
DNLTKAERGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRKVLSMPQVNIVKKTEVQTGG
FSKESILPKRNSDKLIARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLGGD

As used herein, the term “defective CRISPR/Cas nuclease” refers to a CRISPR/Cas nuclease having lost at least one nuclease domain.

As used herein, the term “nickase” has its general meaning in the art and refers to an endonuclease which cleaves only a single strand of a DNA duplex. Accordingly, the term “Cas9 nickase” refers to a nickase derived from a Cas9 protein, typically by inactivating one nuclease domain of Cas9 protein.

As used herein, the term “guide RNA molecule” generally refers to an RNA molecule (or a group of RNA molecules collectively) that can bind to a Cas9 protein and target the Cas9 protein to a specific location within a target DNA. A guide RNA can comprise two segments: a DNA-targeting guide segment and a protein-binding segment. The DNA-targeting segment comprises a nucleotide sequence that is complementary to (or at least can hybridize to under stringent conditions) a target sequence. The protein-binding segment interacts with a CRISPR protein, such as a Cas9 or Cas9 related polypeptide. These two segments can be located in the same RNA molecule or in two or more separate RNA molecules. When the two segments are in separate RNA molecules, the molecule comprising the DNA-targeting guide segment is sometimes referred to as the CRISPR RNA (crRNA), while the molecule comprising the protein-binding segment is referred to as the trans-activating RNA (tracrRNA).

As used herein, the term “target nucleic acid” or “target” refers to a nucleic acid containing a target nucleic acid sequence. A target nucleic acid may be single-stranded or double-stranded, and often is double-stranded DNA. A “target nucleic acid sequence,” “target sequence” or “target region,” as used herein, means a specific sequence or the complement thereof that one wishes to bind to using the CRISPR system as disclosed herein.

As used herein, the term “target nucleic acid strand” refers to a strand of a target nucleic acid that is subject to base-pairing with a guide RNA as disclosed herein. That is, the strand of a target nucleic acid that hybridizes with the crRNA and guide sequence is referred to as the “target nucleic acid strand.” The other strand of the target nucleic acid, which is not complementary to the guide sequence, is referred to as the “non-complementary strand.” In the case of double-stranded target nucleic acid (e.g., DNA), each strand can be a “target nucleic acid strand” to design crRNA and guide RNAs and used to practice the method of this invention as long as there is a suitable PAM site.

As used herein, the term “ribonucleoprotein complex,” or “ribonucleoprotein particle” refers to a complex or particle including a nucleoprotein and a ribonucleic acid. A “nucleoprotein” as provided herein refers to a protein capable of binding a nucleic acid (e.g., RNA, DNA). Where the nucleoprotein binds a ribonucleic acid, it is referred to as “ribonucleoprotein.” The interaction between the ribonucleoprotein and the ribonucleic acid may be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “therapeutically effective amount” is meant a sufficient amount of population of cells to treat the disease at a reasonable benefit/risk ratio applicable to any medical treatment. It will be understood that the total usage compositions of the present invention will be decided by the attending physician within the scope of sound medical judgment. The specific therapeutically effective dose level for any particular patient will depend upon a variety of factors including the age, body weight, general health, sex and diet of the patient, the time of administration, route of administration, the duration of the treatment, drugs used in combination or coincidental with the population of cells, and like factors well known in the medical arts. In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin. A treatment-effective amount of cells in the composition is dependent on the relative representation of the cells with the desired specificity, on the age and weight of the recipient, and on the severity of the targeted condition. This number of cells can be as low as approximately 10³/kg, preferably 5×10³/kg; and as high as 10⁷/kg, preferably 10⁸/kg. The number of cells will depend upon the ultimate use for which the composition is intended, as will the type of cells included therein. Typically, the minimal dose is 2 millions of cells per kg. Usually 2 to 20 millions of cells are injected in the subject. The desired purity can be achieved by introducing a sorting step. For uses provided herein, the cells are generally in a volume of a liter or less, can be 500 ml or less, even 250 ml or 100 ml or less. The clinically relevant number of cells can be apportioned into multiple infusions that cumulatively equal or exceed the desired total amount of cells.

### Methods of the Present Invention

The present invention relates to a method of restoring the normal expression of β-globin in a eukaryotic cell carrying the IVS1-110 (G>A) mutation comprising the step of contacting the eukaryotic cell with a gene editing platform that consists of a (a) at least one adenine base-editor(ABE) and (b) least one guide RNA molecule for guiding the adenine base-editor to at least one target sequence comprising the IVS1-110 (G>A) mutation and thereby restoring the production of β-globin in the eukaryotic cell.

In some embodiments, the eukaryotic cell is selected from the group consisting of hematopoietic progenitor cells, hematopoietic stem cells (HSCs), pluripotent cells (i.e. embryonic stem cells (ES) and induced pluripotent stem cells (iPS)). Typically, the eukaryotic cell results from a stem cell mobilization.

In some embodiments, the eukaryotic cell is homozygous or heterozygous for the IVS1-110 (G>A) mutation.

In some embodiments, the adenine base-editor of the present invention comprises a defective CRISPR/Cas nuclease. The sequence recognition mechanism is the same as for the non-defective CRISPR/Cas nuclease. Typically, the defective CRISPR/Cas nuclease of the invention comprises at least one RNA binding domain. The RNA binding domain interacts with a guide RNA molecule as defined hereinafter. However, the defective CRISPR/Cas nuclease of the invention is a modified version with no nuclease activity. Accordingly, the defective CRISPR/Cas nuclease specifically recognizes the guide RNA molecule and thus guides the base-editor to its target DNA sequence.

In some embodiments, the defective CRISPR/Cas nuclease can be modified to increase nucleic acid binding affinity and/or specificity, alter an enzymatic activity, and/or change another property of the protein. In some embodiments, the nuclease domains of the protein can be modified, deleted, or inactivated. In some embodiments, the protein can be truncated to remove domains that are not essential for the function of the protein. In some embodiments, the protein is truncated or modified to optimize the activity of the RNA binding domain.

In some embodiments, the CRISPR/Cas nuclease consists of a mutant CRISPR/Cas nuclease i.e. a protein having one or more point mutations, insertions, deletions, truncations, a fusion protein, or a combination thereof. In some embodiments, the mutant has the RNA-guided DNA binding activity, but lacks one or both of its nuclease active sites. In some embodiments, the mutant comprises an amino acid sequence having at least 50% of identity with the wild type amino acid sequence of the CRISPR/Cas nuclease. Various CRISPR/Cas nucleases can be used in this invention. Non-limiting examples of suitable CRISPR/CRISPR/Cas nucleases include Cas3, Cas4, Cas5, Cas5e (or CasD), Cas6, Cas6e, Cas6f, Cas7, Cas8al, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmrl, Cmr3, Cmr4, Cmr5, Cmr6, Csb1, Csb2, Csb3, Csxl7, Csxl4, Csx10, Csxl6, CsaX, Csx3, Cszl, Csxl5, Csf1, Csf2, Csf3, Csf4, and Cu1966. See e.g., WO2014144761 WO2014144592, WO2013176772, US20140273226, and US20140273233, the contents of which are incorporated herein by reference in their entireties.

In some embodiments, the CRISPR/Cas nuclease is derived from a type II CRISPR-Cas system. In some embodiments, the CRISPR/Cas nuclease is derived from a Cas9 protein. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonfex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina, inter alia.

In some embodiments, the CRISPR/Cas nuclease is a mutant of a wild type CRISPR/Cas nuclease (such as Cas9) or a fragment thereof. In some embodiments, the CRISPR/Cas nuclease is a mutant Cas9 protein from S. pyogenes.

Methods for generating a Cas9 protein (or a fragment thereof) having an inactive DNA cleavage domain are known (See, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H841A completely inactivate the nuclease activity of S. pyogenes Cas9 (Jinek et al., Science. 337:816-821(2012); Qi et al., Cell. 28; 152(5):1173-83 (2013).

In some embodiments, the CRISPR/Cas nuclease of the present invention is nickase and more particularly a Cas9 nickase i.e. the Cas9 from S. pyogenes having one mutation selected from the group consisting of D10A and H840A. In some embodiments, the nickase of the present invention comprises the amino acid sequence as set forth in SEQ ID NO: 2 or SEQ ID NO:3.

>S. pyogenes nCas9 Protein Sequence having the D10A mutation
SEQ ID NO: 2
MDKKYSIGLAIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEENPINASGVD
AKAILSARLSKSRRLENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNGSIPHQI
HLGELHAILRRQEDFYPFLKDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDFLDNEENEDILEDIVL
TLTLFEDREMIEERLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDELVKVMGRHKPENI
VIEMARENQTTQKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQLLNAKLITQRKF
DNLTKAERGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRKVLSMPQVNIVKKTEVQTGG
FSKESILPKRNSDKLIARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLGGD
>S. pyogenes nCas9 Protein Sequence having the H840A mutation
SEQ ID NO: 3
MDKKYSIGLDIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEENPINASGVD
AKAILSARLSKSRRLENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNGSIPHQI
HLGELHAILRRQEDFYPFLKDNREKIEKILTFRIPYYVGPLARGNSRFAWMTRKSEETITPWNFEEVVD
KGASAQSFIERMTNFDKNLPNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIKDKDFLDNEENEDILEDIVL
TLTLFEDREMIEERLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDELVKVMGRHKPENI
VIEMARENQTTQKGQKNSRERMKRIEEGIKELGSQILKEHPVENTQLQNEKLYLYYLQNGRDMYVDQEL
DINRLSDYDVDAIVPQSFLKDDSIDNKVLTRSDKNRGKSDNVPSEEVVKKMKNYWRQLLNAKLITQRKF
DNLTKAERGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRKVLSMPQVNIVKKTEVQTGG
FSKESILPKRNSDKLIARKKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGNELALPSKYVNFLYLASHY
EKLKGSPEDNEQKQLFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLGGD

In some embodiments, the Cas9 variants having mutations other than D10A or H840A are used, which e.g., result in nuclease inactivated Cas9 (dCas9). Such mutations, by way of example, include other amino acid substitutions at D10 and H840, or other substitutions within the nuclease domains of Cas9 (e.g., substitutions in the HNH nuclease subdomain and/or the RuvC1 subdomain). In some embodiments, variants of dCas9 are provided which are at least about 70% identical, at least about 80% identical, at least about 90% identical, at least about 95% identical, at least about 98% identical, at least about 99% identical, at least about 99.5% identical, or at least about 99.9% to SEQ ID NO: 2 or 3. In some embodiments, variants of dCas9 are provided having amino acid sequences which are shorter, or longer than SEQ ID NO: 2 or 3, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

According to the present invention, the second component of the adenine base-editor herein disclosed comprises a non-nuclease DNA modifying enzyme that is an adenosine deaminase.

In some embodiments, the adenosine deaminase is an ADAT family deaminase. In some embodiments, the adenosine deaminase is a TadA deaminase. In some embodiments, the adenosine deaminase is a Staphylococcus aureus TadA, a Bacillus subtilis TadA, a Salmonella typhimurium TadA, a Shewanella putrefaciens TadA, a Haemophilus influenzae F3031 TadA, a Caulobacter crescentus TadA, or a Geobacter sulfurreducens TadA, or a fragment thereof. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5,6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5,6, 7, 8, 9, 10, 11, 12, 13, 14, 15, 6, 17, 18, 19, or 20C-terminal amino acid residues relative to the full length ecTadA. In some embodiments, the TadA deaminase is TadA*7.10. In some embodiments, the TadA deaminase is a TadA*8 variant. For example, deaminase are described in International PCT Application WO2018/027078, WO2017/070632, WO/2020/168132, WO/2021/050571 each of which is incorporated herein by reference for its entirety. Also, see Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); Komor, A. C., et al., “Improved base excision repair inhibition and bacteriophage Mu Gam protein yields C:G-to-T:A base editors with higher efficiency and product purity” Science Advances 3:eaao4774 (2017)), and Rees, H. A., et al., “Base editing: precision chemistry on the genome and transcriptome of living cells.” Nat Rev Genet. 2018 Dec.; 19(12):770-788. doi: 10.1038/s41576-018-0059-1, the entire contents of which are hereby incorporated by reference. An exemplary amino acid sequence for the wild type TadA(wt) adenosine deaminase is shown as SEQ ID NO:4. In some embodiments, the amino acid sequence of the adenosine deaminase comprises at least 90% sequence identity to SEQ ID NO:4. In some embodiments, the amino acid sequence of the adenosine deaminase comprises the modification at position 82 as numbered in SEQ ID NO:4. In some embodiments, the amino acid sequence comprises of the adenosine deaminase comprises a V82S modification, wherein position 82 is as numbered in SEQ ID NO:4. In some embodiments, the amino acid sequence of the adenosine deaminase comprises the modification at position 166 as numbered in SEQ ID NO:4. In some embodiments, the amino acid sequence of the adenosine deaminase comprises a T166R modification, wherein position 166 is as numbered in SEQ ID NO:4. In some embodiments, the amino acid sequence of the adenosine deaminase comprises modifications at positions 82 and 166 as numbered in SEQ ID NO:4. In some embodiments, the amino acid sequence of the adenosine deaminase comprises V82S and T166R modifications, wherein positions 82 and 166 are as numbered in SEQ ID NO:4. In some embodiments, the adenosine deaminase variant further comprises one or more of the following alterations: Y147T, Y147R, Q154S, Y123H, and Q154R. In some embodiments, the adenosine deaminase variant comprises a combination of alterations selected from the group consisting of: Y147T+Q154R; Y147T+Q154S; Y147R +Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+176Y; V82S+Y123H+Y147R+Q154R; and T76Y+V82S+Y123H+Y147R+Q154R. In some embodiments, the adenosine deaminase variant is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24. In some embodiments, the adenosine deaminase is provided as a single (e.g., provided as a monomer) TadA variant as described above. In some embodiments, adenosine deaminase is provided as a heterodimer of a wild-type TadA (TadA(wt)) linked to a TadA variant as described above.

>TadA sequence
SEQ ID NO: 4
MSEVEFSHEYWMRHALTLAKRAWDEREVPVGAVLVHNNRVIGEGWNRPIGRHDPTAHAEIMALRQGGLV
MQNYRLIDATLYVTLEPCVMCAGAMIHSRIGRVVFGARDAKTGAAGSLMDVLHHPGMNHRVEITEGILA
DECAALLSDFFRMRRQEIKAQKKAQSSTD

In some embodiments, the adenosine deaminase is fused to the N-terminus of the defective CRISPR/Cas nuclease. In some embodiments, the adenosine deaminase is fused to the C-terminus of the defective CRISPR/Cas nuclease. In some embodiments, the defective CRISPR/Cas nuclease and the adenosine deaminase are fused via a linker. In some embodiments, the linker comprises a (GGGGS)n (SEQ ID NO:5), a (G)n, an (EAAAK)n (SEQ ID NO:6), a (GGS)n, an SGSETPGTSESATPES (SEQ ID NO:7) motif (see, e.g., Guilinger J P, Thompson D B, Liu D R. Additional suitable linker motifs and linker configurations will be apparent to those of skill in the art. In some embodiments, suitable linker motifs and configurations include those described in Chen et al., Fusion protein linkers: property, design and functionality. Adv Drug Deliv Rev. 2013; 65(10):1357-69, the entire contents of which are incorporated herein by reference.

In some embodiments, the fusion protein may comprise additional features. Other exemplary features that may be present are localization sequences, such as nuclear localization sequences (NLS), cytoplasmic localization sequences, export sequences, such as nuclear export sequences, or other localization sequences, as well as sequence tags that are useful for solubilization, purification, or detection of the fusion proteins. Suitable localization signal sequences and sequences of protein tags are provided herein, and include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable features will be apparent to those of skill in the art.

Various adenine base-editors are known in the art (see e.g. Improving cytidine and adenine base-editors by expression optimization and ancestral reconstruction. Nat Biotechnol. 2018 May 29) and typically include those described in Table A.

TABLE A
some exemplary base-editors

| Base- | |
|---|---|
| editor | References |
| ABEmax | Improving cytidine and adenine base-editors by expression |
| | optimization and ancestral reconstruction. Nat Biotechnol. |
| | 2018 May 29. pii: nbt.4172. doi: 10.1038/nbt.4172. |
| ABE- | Walton R T, Christie K A, Whittaker M N, Kleinstiver B P. |
| SpRY | Unconstrained genome targeting with near-PAMless |
| | engineered CRISPR-Cas9 variants. Science. 2020 Apr. 17; |
| | 368(6488): 290-296. doi: 10.1126/science.aba8853. Epub |
| | 2020 Mar. 26. PMID: 32217751; PMCID: PMC7297043. |
| ABE8e | Richter, M. F., Zhao, K. T., Eton, E. et al. Phage-assisted |
| | evolution of an adenine base editor with improved Cas domain |
| | compatibility and activity. Nat Biotechnol 38, 883-891 (2020). |

In some embodiments, the adenine base-editor consists of the amino acid sequence as set forth in ID NO:8 (SpRY-ABE8e).

>amino acid sequence of SpRY-ABE8e
SEQ ID NO: 8
MKRTADGSEFESPKKKRKVSEVEFSHEYWMRHALTLAKRARDEREVPVGAVLVLNNRVIGEGWNRAIGL
HDPTAHAEIMALRQGGLVMQNYRLIDATLYVTFEPCVMCAGAMIHSRIGRVVFGVRNSKRGAAGSLMNV
LNYPGMNHRVEITEGILADECAALLCDFYRMPRQVENAQKKAQSSINSGGSSGGSSGSETPGTSESATP
ESSGGSSGGSDKKYSIGLAIGTNSVGWAVITDEYKVPSKKFKVLGNTDRHSIKKNLIGALLFDSGETAE
RTRLKRTARRRYTRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQLVQTYNQLFEE
NPINASGVDAKAILSARLSKSRRLENLIAQLPGEKKNGLFGNLIALSLGLTPNFKSNEDLAEDAKLQLS
KDTYDDDLDNLLAQIGDQYADLFLAAKNLSDAILLSDILRVNTEITKAPLSASMIKRYDEHHQDLTLLK
ALVRQQLPEKYKEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAWMTRKSEETIT
PWNFEEVVDKGASAQSFIERMTNFDKNLPNEKVLPKHSLLYEYFTVYNELTKVKYVTEGMRKPAFLSGE
QKKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTILD
FLKSDGFANRNFMQLIHDDSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVENTQLQNEKLYLYYLQNG
RDMYVDQELDINRLSDYDVDHIVPQSFLKDDSIDNKVLTRSDKNRGKSDNVPSEEVVKKMKNYWRQLLN
AKLITQRKFDNLTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVRKVLSMPQVNIV
KKTEVQTGGFSKESIRPKRNSDKLIARKKDWDPKKYGGFLWPTVAYSVLVVAKVEKGKSKKLKSVKELL
GITIMERSSFEKNPIDFLEAKGYKEVKKDLIIKLPKYSLFELENGRKRMLASAKQLQKGNELALPSKYV
NFLYLASHYEKLKGSPEDNEQKQLFVEQHKHYLDEIIEQISEFSKRVILADANLDKVLSAYNKHRDKPI
REQAENIIHLFTLTRLGAPRAFKYFDTTIDPKQYRSTKEVLDATLIHQSITGLYETRIDLSQLGGDSGG
SKRTADGSEFEPKKKRKVGSGATNFSLLKQAGDVEENPGPMVSKGEELFTGVVPILVELDGDVNGHKFS
VSGEGEGDATYGKLTLKFICTTGKLPVPWPTLVTTLTYGVQCFSRYPDHMKQHDFFKSAMPEGYVQERT
IFFKDDGNYKTRAEVKFEGDTLVNRIELKGIDFKEDGNILGHKLEYNYNSHNVYIMADKQKNGIKVNFK
IRHNIEDGSVQLADHYQQNTPIGDGPVLLPDNHYLSTQSALSKDPNEKRDHMVLLEFVTAAGITLGMDE
LYKSGGSPKKKRKV*

The second component of the gene-editing platform disclosed herein consists of at least one guide RNA molecule suitable for guiding the base-editor to at least one target sequence that comprises the IVS1-110 (G>A) mutation. The guide RNA molecule of the present invention thus comprises a guide sequence for providing the targeting specificity. It includes a region that is complementary and capable of hybridization to a pre-selected target site of interest.

In some embodiment, this guide sequence can comprise from about 10 nucleotides to more than about 25 nucleotides. For example, the region of base pairing between the guide sequence and the corresponding target site sequence can be about 10, 11, 12, 13, 14, 15, 16, 17, 18, 19, 20, 22, 23, 24, 25, or more than 25 nucleotides in length. In some embodiments, the guide sequence is about 17-20 nucleotides in length, such as 20 nucleotides.

Typically, a software program is used to identify candidate CRISPR target sequences on both strands of the DNA nucleic acid molecule based on desired guide sequence length and a CRISPR motif sequence (PAM) for a specified CRISPR enzyme. One requirement for selecting a suitable target nucleic acid is that it has a 3′ PAM site/sequence. Each target sequence and its corresponding PAM site/sequence are referred herein as a Cas-targeted site. Type II CRISPR system, one of the most well characterized systems, needs only Cas 9 protein and a guide RNA complementary to a target sequence to affect target cleavage. For example, target sites for Cas9 from S. pyogenes, with PAM sequences NGG, may be identified by searching for 5′-Nx-NGG-3′ both on the input sequence and on the reverse-complement of the input. Since multiple occurrences in the genome of the DNA target site may lead to nonspecific genome editing, after identifying all potential sites, the program filters out sequences based on the number of times they appear in the relevant reference genome. For those CRISPR enzymes for which sequence specificity is determined by a “seed” sequence, such as the 11-12 bp 5′ from the PAM sequence, including the PAM sequence itself, the filtering step may be based on the seed sequence. Thus, to avoid editing at additional genomic loci, results are filtered based on the number of occurrences of the seed:PAM sequence in the relevant genome. The user may be allowed to choose the length of the seed sequence. The user may also be allowed to specify the number of occurrences of the seed:PAM sequence in a genome for purposes of passing the filter. The default is to screen for unique sequences. Filtration level is altered by changing both the length of the seed sequence and the number of occurrences of the sequence in the genome. The program may in addition or alternatively provide the sequence of a guide sequence complementary to the reported target sequence(s) by providing the reverse complement of the identified target sequence(s). Further details of methods and algorithms to optimize sequence selection can be found in U.S. application Ser. No. 61/836,080; incorporated herein by reference.

In some embodiments, the guide RNA targets a sequence selected from Table 1 (see EXAMPLE).

In some embodiments, the gene editing platform comprises a) the adenine base-editor SpRY-ABE8e and b) and at least one gRNA molecule that targets a sequence selected from Table 1.

The guide RNA molecule of the present invention can be made by various methods known in the art including cell-based expression, in vitro transcription, and chemical synthesis. The ability to chemically synthesize relatively long RNAs (as long as 200 mers or more) using TC-RNA chemistry (see, e.g., U.S. Pat. No. 8,202,983) allows one to produce RNAs with special features that outperform those enabled by the basic four ribonucleotides (A, C, G and U). In particular, the RNA molecule of the present invention can be made with recombinant technology using a host cell system or an in vitro translation-transcription system known in the art. Details of such systems and technology can be found in e.g., WO2014144761 WO2014144592, WO2013176772, US20140273226, and US20140273233, the contents of which are incorporated herein by reference in their entireties.

In some embodiments, the guide RNA molecule may include one or more modifications. Such modifications may include inclusion of at least one non-naturally occurring nucleotide, or a modified nucleotide, or analogs thereof. Modified nucleotides may be modified at the ribose, phosphate, and/or base moiety. Modified nucleotides may include 2′-O-methyl analogs, 2′-deoxy analogs, or 2′-fluoro analogs. The nucleic acid backbone may be modified, for example, a phosphorothioate backbone may be used. The use of locked nucleic acids (LNA) or bridged nucleic acids (BNA) may also be possible. Further examples of modified bases include, but are not limited to, 2-aminopurine, 5-bromo-uridine, pseudouridine, inosine, 7-methylguanosine.

In some embodiments, the different components of the gene editing platform of the present invention are provided to the eukaryotic cell through expression from one or more expression vectors. For example, the nucleic acids encoding the guide RNA molecule or the base-editor can be cloned into one or more vectors for introducing them into the eukaryotic cell. The vectors are typically prokaryotic vectors, e.g., plasmids, or shuttle vectors, or insect vectors, for storage or manipulation of the nucleic acid encoding the guide RNA molecule or the base-editor herein disclosed. Preferably, the nucleic acids are isolated and/or purified. Thus, the present invention provides recombinant constructs or vectors having sequences encoding one or more of the guide RNA molecule or base-editors described above. Examples of the constructs include a vector, such as a plasmid or viral vector, into which a nucleic acid sequence of the invention has been inserted, in a forward or reverse orientation. In some embodiments, the construct further includes regulatory sequences. A “regulatory sequence” includes promoters, enhancers, and other expression control elements (e.g., polyadenylation signals). Regulatory sequences include those that direct constitutive expression of a nucleotide sequence, as well as inducible regulatory sequences. The design of the expression vector can depend on such factors as the choice of the eukaryotic cell to be transformed, transfected, or infected, the desired expression level, and the like. Large numbers of suitable vectors and promoters are known to those of skill in the art, and are commercially available. Appropriate cloning and expression vectors for use with eukaryotic hosts are also described in e.g., Sambrook et al. (2001, Molecular Cloning: A Laboratory Manual, Cold Spring Harbor Press). The vector can be capable of autonomous replication or integration into a host DNA. The vector may also include appropriate sequences for amplifying expression. In addition, the expression vector preferably contains one or more selectable marker genes to provide a phenotypic trait for selection of transformed host cells such as dihydrofolate reductase or neomycin resistance for eukaryotic cell cultures, or such as tetracycline or ampicillin resistance in E. coli. Any of the procedures known in the art for introducing foreign nucleotide sequences into host cells may be used. Examples include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell.

In some embodiments, the different components of the gene editing platform of the present invention are provided to the population of cells through the use of an RNA-encoded system. In particular, the base-editing system may be provided to the population of cells through the use of a chemically modified mRNA-encoded adenine or cytidine base editor together with modified guide RNA as described in Jiang, T., Henderson, J. M, Coote, K. et al. Chemical modifications of adenine base editor mRNA and guide RNA expand its application scope. Nat Commun 11, 1979 (2020). In particular, engineered RNA-encoded base-editors (e.g. ABE) system are prepared by introducing various chemical modifications to both mRNA that encoded the base-editor and guide RNA. In particular said modifications consist in uridine depleted mRNAs modified with 5-methoxyuridine: synonymous codons may be introduced to deplete uridines as much as possible without altering the coding sequence and replaced all the remaining uridines with 5-methoxyuridine. Said optimized base editing system exhibits higher editing efficiency at some genomic sites compared to DNA-encoded system. It is also possible to encapsulate the modified mRNA and guide RNA into lipid nanoparticle (LNP) for allowing lipid nanoparticle (LNP)-mediated delivery.

In some embodiments, the different components of the gene editing platform of the present invention are provided to the population of cells through the use of ribonucleoprotein (RNP) complexes. For instance. the base-editor can be pre-complexed with one or more guide RNA molecules to form a ribonucleoprotein (RNP) complex. The RNP complex can thus be introduced into the eukaryotic cell. Introduction of the RNP complex can be timed. The cell can be synchronized with other cells at G1, S, and/or M phases of the cell cycle. RNP delivery avoids many of the pitfalls associated with mRNA, DNA, or viral delivery. Typically, the RNP complex is produced simply by mixing the proteins (i.e. the base-editor) and one or more guide RNA molecules in an appropriate buffer. This mixture is incubated for 5-10 min at room temperature before electroporation. Electroporation is a delivery technique in which an electrical field is applied to one or more cells in order to increase the permeability of the cell membrane. In some embodiments, genome editing efficiency can be improved by adding a transfection enhancer oligonucleotide.

In some embodiments, a plurality of successive transfections are performed for reaching a desired level of mutagenesis in the cell.

A further object of the present invention relates to a method of treating β-thalassemia in a subject in need thereof, the method comprising transplanting a therapeutically effective amount of a population of eukaryotic cells obtained by the method as above described.

In some embodiments, the population of eukaryotic cells is autologous to the subject, meaning the population of cells is derived from the same subject.

In some embodiments, the patient suffers from sickle β-thalassemia.

### Kits

This invention further provides kits containing reagents for performing the above-described methods, including all component of the gene editing platform as disclosed herein for performing mutagenesis. To that end, one or more of the reaction components, e.g., guide RNA molecules, and nucleic acid molecules encoding for the base-editors for the methods disclosed herein can be supplied in the form of a kit for use. In some embodiments, the kit comprises one or more base-editors and one or more guide RNA molecules. In some embodiments, the kit can include one or more other reaction components. In some embodiments, an appropriate amount of one or more reaction components is provided in one or more containers or held on a substrate. Examples of additional components of the kits include, but are not limited to, one or more host cells, one or more reagents for introducing foreign nucleotide sequences into host cells, one or more reagents (e.g., probes or PCR primers) for detecting expression of the guide RNA or base-editors or verifying the target nucleic acid's status, and buffers or culture media for the reactions. The kit may also include one or more of the following components: supports, terminating, modifying or digestion reagents, osmolytes, and an apparatus for detection. The components used can be provided in a variety of forms. For example, the components (e.g., enzymes, RNAs, probes and/or primers) can be suspended in an aqueous solution or as a freeze-dried or lyophilized powder, pellet, or bead. In the latter case, the components, when reconstituted, form a complete mixture of components for use in an assay. The kits of the invention can be provided at any suitable temperature. For example, for storage of kits containing protein components or complexes thereof in a liquid, it is preferred that they are provided and maintained below 0° C., preferably at or below −20° C., or otherwise in a frozen state. The kits can also include packaging materials for holding the container or combination of containers. Typical packaging materials for such kits and systems include solid matrices (e.g., glass, plastic, paper, foil, micro-particles and the like) that hold the reaction components or detection probes in any of a variety of configurations (e.g., in a vial, microtiter plate well, microarray, and the like). The kits may further include instructions recorded in a tangible form for use of the components.

The invention will be further illustrated by the following figures and examples. However, these examples and figures should not be interpreted in any way as limiting the scope of the present invention.

### FIGURES

FIG. 1. Design and screening of gRNAs targeting the IVS1-110 (G>A) mutation in β-thalassemic T cells.

A. gRNAs1-6 were manually designed to place the IVS1-110 (G>A) mutation in position 3 to 8 of the editing window. The mutation is highlighted with a grey box. B. Overview of the cell collection for testing the ability of gRNA/BE to revert the IVS1-110 (G>A) mutation. Peripheral blood mononuclear cells (PBMCs) were isolated from 2 homozygous and 1 compound heterozygous thalassemia patients harboring the IVS1-110 (G>A) mutation. After CD34⁺ cell sorting, T cells were recovered from the negative fraction for testing gRNA/BE combinations, before moving to CD34⁺ cells with a selected strategy. C. Frequency of corrected alleles (normalized to the frequency of GFP⁺ cells) as evaluated by EditR and InDel frequency as assessed by TIDE in T cells transfected with different combinations of synthetic gRNAs and ABE mRNAs. Data are expressed as mean±standard error of the mean (SEM) (n=2 biologically independent experiments, 2 donors). D. Representative percent composition of Sanger sequencing traces measured to be significantly different from noise (in grey), as assessed by EditR following Sanger sequencing in T cells transfected with gRNA1/ABE8e-SpRY. Target base position is highlighted with a black box and the bystander edits with grey boxes.

FIG. 2. Efficient correction of the IVS1-110 (G>A) mutation in β-thalassemic HSPCs restores normal Hb production.

A. Experimental protocol used for base editing experiments in β-thalassemic HSPCs. SpRY-ABEe mRNA and a synthetic gRNA were co-transfected in β-thalassemic HSPCs. Cells were differentiated into mature RBCs using a three-phase erythroid differentiation protocol. B. Frequency of corrected alleles as evaluated by EditR and InDel frequency as assessed by TIDE in β-thalassemic HSPCs. Data are expressed as mean±SEM (n=2 biologically independent experiments, 3 donors). Frequency of corrected alleles in the cells from the compound heterozygous patient (BT2) was corrected to take into account only alleles harbouring the IVS1-110 (G>A) mutation. C. Percent composition of Sanger sequencing traces measured to be significantly different from noise (in grey), as assessed by EditR following Sanger sequencing in CD34⁺ cells transfected with gRNA1/ABE8e-SpRY. Target base position is highlighted with a black box and the bystander edits with grey boxes. D. Expression of -β, ^{G}γ-, ^{A}γ- and δ-globin chains measured by RP-HPLC in β-thalassemic patient and healthy donor RBCs. β-like-globin expression was normalized to α-globin. The ratio α/non-α globins is reported on top of the graph. RBCs were obtained from corrected β-thalassemic HSPCs (cor). As controls, we used RBCs derived from β-thalassemic patients' or healthy donor HSPCs transfected with TE (TE) or only with SpRY-ABEe mRNA (BE) (n=2 biologically independent experiments, 3 β-thalassemia patients and 2 healthy donors). Data are expressed as mean±SEM. E. Analysis of HbA, HbF and HbA₂ by CE-HPLC in β-thalassemic patient and healthy donor RBCs. We calculated the percentage of each Hb type over the total Hb tetramers. RBCs were obtained from corrected β-thalassemic HSPCs (cor). As controls, we used RBCs derived from β-thalassemic patients' or healthy donor HSPCs transfected with TE (TE) or only with SpRY-ABEe mRNA (BE) (n=2 biologically independent experiments, 3 β-thalassemia patients and 2 healthy donors). Data are expressed as mean±SEM. BT, β-thalassemia patients. HD, healthy donors.

FIG. 3. Efficient reversion of the IVS1-110 (G>A) mutation in β-thalassemic HSPCs corrects ineffective erythropoiesis.

A-C. Frequency of GPA⁺ (A), CD36⁺ (B) and CD71⁺ (C) cells at day 13, 16 and 20 of erythroid differentiation, as measured by flow cytometry analysis. Data are expressed as mean±SEM (n=2 biologically independent experiments, 3 β-thalassemia patients and 3 healthy donors). D. Frequency of enucleated cells at day 13, 16 and 20 of erythroid differentiation, as measured by flow cytometry analysis of cells stained with the DRAQ5 nuclear dye (n=2 biologically independent experiments, 3 β-thalassemia patients and 3 healthy donors). Data for HD samples are expressed as mean±SEM. E. Cell size of enucleated cells at day 13, 16 and 20 of erythroid differentiation, as measured by flow cytometry using the median of FSC-A intensity. (n=2 biologically independent experiments, 3 β-thalassemia patients and 3 healthy donors). Data for HD samples are expressed as mean±SEM. F. Flow cytometry histograms showing the frequency of apoptotic cells (AnnexinV⁺-cells) in the 7AAD⁻ cell population in unstained (Uns), β-thalassemic and healthy donor samples at day 13 of erythroid differentiation (n=2 biologically independent experiments, 3 β-thalassemia patients and 3 healthy donors).

FIG. 4. Correction of the IVS1-110 (G>A) mutation in repopulating HSCs.

A. Overview of the experimental protocol of HSPC xenotransplantation. BT HSPCs were subjected to RNA-mediated base editing and xenotransplanted into NBSGW immunodeficient mice. HD and β-thalassemic HSPCs transfected with TE buffer or only with SpRY-ABE8e mRNA were injected as controls. Peripheral blood analysis was performed at weeks 9 and 16. Mice were euthanized 16 weeks after engraftment, following Clo-Lip injection, and hematopoietic tissues and organs were collected and analyzed. B. Engraftment of human cells in NBSGW mice transplanted with HD or BT control (HD-ctr, BT-ctr) or corrected (BT-cor) HSPCs 16 weeks post-transplantation (HD-ctr: n=4; BT-ctr: n=4; BT-cor: n=5). Engraftment is represented as percentage of human CD45+ cells in the total murine and human CD45⁺ cell population in peripheral blood, bone marrow, spleen and thymus. Each data point represents an individual mouse. The mouse with a lowest chimerism is indicated with the symbol [symbol] . Data are expressed as mean±SEM. C. Frequency of human T (CD3) and B (CD19) lymphoid, myeloid (CD14, CD15 and CD11b), erythroid (GPA, CD36, CD71) and HSPC (CD34) cells in BM 16 weeks post-transplantation (HD-ctr: n=4; BT-ctr: n=4; BT-cor: n=5). Each data point represents an individual mouse. Data are expressed as mean±SEM. D. Human hematopoietic progenitor content in BM human CD45⁺ cells derived from mice transplanted with control and edited HSPCs (HD-ctr: n=4; BT-ctr: n=4; BT-cor: n=5). We plotted the percentage of human CD45⁺ cells giving rise to BFU-E and CFU-GM. Data are expressed as mean±SEM. E. Base editing efficiency, calculated by the EditR software, in input, peripheral blood-, bone marrow- and spleen-derived HD and BT human samples subjected to Sanger sequencing. Data are expressed as mean±SEM (BT-cor: n=5). The frequency of base editing in the input was calculated in cells cultured in the HSPC medium (▴), in liquid erythroid cultures (▾), BFU-E (▪) and CFU-GM (♦) colonies. Each data point represents an individual mouse. Data are expressed as mean±SEM.

FIG. 5. Correction of the IVS1-110 (G>A) mutation in xenotransplanted β-thalassemic HSPCs rescues the ineffective erythropoiesis in vivo.

A. Frequency of ROS-containing (DCFDA⁺) human GPA⁺ erythroid cells derived from the bone marrow of mice transplanted with HD or BT control (HD-ctr, BT-ctr) or corrected (BT-cor) HSPCs 16 weeks post-transplantation (HD-ctr: n=3; BT-ctr: n=4; BT-cor: n=4). ** p≤0.01 (Unpaired T-test, BT-ctr vs BT-cor). We plotted the fold change relative to BT-ctr samples. B. Frequency of enucleated cells as measured by flow cytometry analysis of cells stained with the DRAQ5 nuclear dye in human GPA⁺ erythroid populations from the bone marrow of mice transplanted with HD or BT control (HD-ctr, BT-ctr) or corrected (BT-cor) HSPCs 16 weeks post-transplantation (HD-ctr: n=3; BT-ctr: n=4; BT-cor: n=4). * p≤0.05 (Unpaired T-test, BT-ctr vs BT-cor). C. Representative RP-HPLC chromatograms from sorted human GPA* bone marrow erythroid cells 16 weeks post-transplantation. D. a/non-α ratio calculated based on RP-HPLC data from sorted human GPA* bone marrow erythroid cells obtained from mice transplanted with HD or BT control (HD-ctr, BT-ctr) or corrected (BT-cor) HSPCs 16 weeks post-transplantation (HD-ctr: n=4; BT-ctr: n=4; BT-cor: n=5). Dotted lines indicate minimum and maximum values observed in HD-ctr samples. * p≤0.05 (Unpaired T-test, BT-ctr vs BT-cor). E. Frequency of human RBCs in total peripheral blood 4 days after Clo-Lip injection in mice transplanted with HD or BT control (HD-ctr, BT-ctr) or corrected (BT-cor) HSPCs 16 weeks post-transplantation (HD-ctr: n=4; BT-ctr: n=4; BT-cor: n=5). White dots indicate the mice from which peripheral blood human GPA* cells were sorted for subsequent HPLC analysis. F. Representative RP-HPLC chromatograms from sorted human circulating RBCs 16 weeks post-transplantation (BT-ctr: n=4 pooled samples; BT-cor: n=1 representative graph; HD-ctr: n=1 representative graph).

### Example

### Material & Methods

### HSPC and T Cell Purification and Culture

We obtained human peripheral blood non-mobilized CD34⁺ HSPCs from β-thalassemia patients (for in vitro experiments). HD CD34⁺ HSPCs samples were obtained from bone marrow of healthy donors (for in vitro experiments), or obtained following GCSF mobilization (for in vivo experiments). Written informed consent was obtained from all subjects. All experiments were performed in accordance with the Declaration of Helsinki. The study was approved by the regional investigational review board (reference: DC 2014-2272, CPP Ile-de-France II “H6pital Necker-Enfants malades”). HSPCs were purified using the CD34 MicroBead Kit (Miltenyi Biotec).

Plerixafor/GCSF-mobilized peripheral blood CD34⁺ cells (used for in vivo experiments) were selected from a patient affected by β-thalassemia upon signed informed consent approved by the Ethical Committee of the San Raffaele Hospital (Milan, Italy). Following mobilization and cell collection with the Spectra Cobe or Spectra Optia apheresis system (Terumo BCT), CD34⁺ cells were purified using immunomagnetic beads (CliniMACS, Miltenyi Biotec) by MolMed SpA (Milan, Italy).

Forty-eight hours before transfection, CD34+ cells were thawed and cultured at a concentration of 5×10⁵ cells/mL in “HSPC medium” containing StemSpan (STEMCELL Technologies) supplemented with penicillin/streptomycin (Gibco), StemRegenin1 (STEMCELL Technologies) and the following recombinant human cytokines (PeproTech): human stem cell factor, Fms-like tyrosine kinase receptor 3 ligand, thrombopoietin and interleukin-3. Four days before transfection, the CD34⁻ fraction was thawed and cultured at a concentration of 5×10⁶ cells/mL in “T cell medium” containing RPMI 1640+ GlutaMAX (Gibco) supplemented with FBS (Thermo), penicillin/streptomycin (Gibco) and recombinant human interleukin-2 (Peprotech). Five hours after thawing, cells were transferred to “T cell activation medium” containing RPMI 1640+GlutaMAX (Gibco) supplemented with CD28 Monoclonal Antibody (eBioscience, Clone CD28.2) in plates coated with CD3 Monoclonal Antibody (eBioscience, Clone OKT3).

### Base Editor Plasmids

Plasmids used in this study include NG-ABE8e (Plasmid #138491, Addgene), ABE8e (Plasmid #138489, Addgene) and pCMV-T7-ABEmax(7.10)-SpRY-P2A-EGFP (RTW5025) (Plasmid #140003, Addgene). The ABE8e-SpRY plasmid was created by replacing the Cas9 coding sequence of the ABE8e plasmid by the one fused to GFP included in the “pCMV-T7-ABEmax(7.10)-SpRY-P2A-EGFP (RTW5025)” plasmid.

gRNA Design

We manually designed gRNAs targeting the IVS1-110 (G>A) mutation (Table 1). We used chemically modified synthetic gRNAs harboring 2′-O-methyl analogs and 3′-phosphorothioate nonhydrolyzable linkages at the first three 5′ and 3′ nucleotides (Synthego).

TABLE 1
gRNA target sequences.

| | Sequence | PAM sequence | Position | | Compatible |
|---|---|---|---|---|---|
| gRNA | (5′ to 3′) | (5′ to 3′) | (hg19) | Strand | ABEs |
| gRNA1 | TTAGTCTATTTTCCCACCCT | TAG | chr11: 5248033- | − | SpRY-ABE8e, NG- |
| | (SEQ ID NO: 9) | | 5248052 (HBB) | | ABE8e, ABE8e |
| gRNA2 | ATTAGTCTATTTTCCCACCC | TTA | chr11: 5248034- | − | SpRY-ABE8e |
| | (SEQ ID NO: 10) | | 5248053 (HBB) | | |
| gRNA3 | TATTAGTCTATTTTCCCACC | CTT | chr11: 5248035- | − | SpRY-ABE8e |
| | (SEQ ID NO: 11) | | 5248054 (HBB) | | |
| gRNA4 | CTATTAGTCTATTTTCCCAC | CCT | chr11: 5248036- | − | SpRY-ABE8e |
| | (SEQ ID NO: 12) | | 5248055 (HBB) | | |
| gRNA5 | CCTATTAGTCTATTTTCCCA | CCC | chr11: 5248037- | − | SpRY-ABE8e |
| | (SEQ ID NO: 13) | | 5248056 (HBB) | | |
| gRNA6 | GCCTATTAGTCTATTTTCCC | ACC | chr11: 5248038- | − | SpRY-ABE8e |
| | (SEQ ID NO: 14) | | 5248057 (HBB) | | |

mRNA In Vitro Transcription

20 μg of NG-ABE8e, ABE8e or SpRY-ABE8e expressing plasmids were digested overnight with SapI restriction enzyme (Thermo) that cleaves once right after the poly-A tail. The linearized plasmids were purified using a PCR purification kit (QIAGEN #28106) and were eluted in 14 μl of DNase/RNase-free water. 2 g of linearized plasmid were used as template for the in vitro transcription reaction (MEGAscript, Ambion #AM1334). The in vitro transcription protocol was modified as follows. The GTP nucleotide solution was used at a final concentration of 3.0 mM instead of 7.5 mM and the anti-reverse cap analog N7-Methyl-3′-O-Methyl-Guanosine-5′-Triphosphate-5′-Guanosine (ARCA, Trilink #N-7003) was used at a final concentration of 12.0 mM resulting in a final ratio of Cap:GTP of 4:1 that allows efficient mRNA capping. The incubation time for the in vitro reaction was reduced to 30 minutes. mRNA was precipitated using lithium chloride and resuspended in TE buffer in a final volume that allowed to achieve a concentration of >1 μg/l. The mRNA quality was assessed using Bioanalyzer (Agilent).

### RNA Transfection

1×10⁶ T cells per condition were transfected with 3.0 μg of the ABE-encoding mRNA and 3.2 μg of the synthetic gRNA. When ABE was not fused to GFP, a GFP-encoding mRNA (Tebu-bio) was added to the transfection mix. We used the P3 Primary Cell 4D-Nucleofector X Kit S (Lonza) and the E0115 program (Nucleofector 4D). Cells transfected only with TE buffer served as negative controls.

1×10⁴ to 5×10⁵ HSPCs per condition were transfected with 3.0 μg of the ABE-encoding mRNA and 3.2 μg of the synthetic gRNA. We used the P3 Primary Cell 4D-Nucleofector X Kit S (Lonza) and the CA137 program (Nucleofector 4D). For in vivo experiments, 2.3 to 7.5×10⁶ HSPCs per condition were transfected with 15.0 g of the ABE-encoding mRNA and 16.0 g of the synthetic gRNA using the P3 Primary Cell 4D-Nucleofector X Kit L (Lonza) and the CA-137 program (Nucleofector 4D). Cells transfected only with TE buffer or only with the ABE-encoding mRNA served as negative controls.

### HSPC Differentiation

Transfected CD34⁺ HSPCs were differentiated into mature red blood cells (RBCs) using a three-phase erythroid differentiation protocol, as previously described^{23,24}. During the first phase (day 0 to day 6), cells were cultured in a basal erythroid medium supplemented with 100 ng/ml recombinant human SCF (PeproTech), 5 ng/ml recombinant human IL-3 (PeproTech), 3 IU/ml EPO Eprex (Janssen-Cilag) and 10⁻⁶ M hydrocortisone (Sigma). During the second phase (day 6 to day 9), cells were co-cultured with MS-5 stromal cells in the basal erythroid medium supplemented with 3 IU/ml EPO Eprex (Janssen-Cilag). During the third phase (day 9 to day 20), cells were co-cultured with stromal MS-5 cells in a basal erythroid medium without cytokines. Erythroid differentiation was monitored by flow cytometry analysis of CD36, CD71, GYPA and of enucleated cells using the DRAQ5 double-stranded DNA dye. 7AAD was used to identify live cells.

### Evaluation of Editing Efficiency

Base editing efficiency and InDel frequency were evaluated in HSPC-derived erythroid cells at the end of the second phase of differentiation. Genomic DNA was extracted from control and edited cells using PURE LINK Genomic DNA Mini kit (LifeTechnologies), or Quick-DNA/RNA Miniprep (ZYMO Research), following manufacturers' instructions. To evaluate base editing efficiency at gRNA target sites, we performed a nested PCR using previously published primers¹⁰, followed by Sanger sequencing and EditR analysis²⁵. TIDE analysis (Tracking of InDels by Decomposition) was performed to evaluate the percentage of InDels in edited samples²⁶.

### Flow Cytometry Analysis

Flow cytometry analysis of CD36, CD71 and GYPA erythroid surface markers on HSPC-derived erythroid cells was performed using a V450-conjugated anti-CD36 antibody (561535, BD Horizon), a FITC-conjugated anti-CD71 antibody (555536, BD Pharmingen) and a PE-Cy7-conjugated anti-GYPA antibody (563666, BD Pharmingen). Flow cytometry analysis of enucleated or viable cells was performed using double-stranded DNA dyes (DRAQ5, 65-0880-96, Invitrogen and 7AAD, 559925, BD, respectively). Apoptosis was evaluated using PE Annexin V Apoptosis Detection Kit I (BD Biosciences). Flow cytometry analyses were performed using Gallios (Beckman coulter) or Novocyte (Agilent) flow cytometer. Data were analyzed using the FlowJo (BD Biosciences) software.

### RP-HPLC Analysis of Globin Chains

Reversed-phase HPLC analysis was performed using a NexeraX2 SIL-30AC chromatograph and the LC Solution software (Shimadzu). A 250×4.6 mm, 3.6 μm Aeris Widepore column (Phenomenex) was used to separate globin chains by HPLC. Samples were eluted with a gradient mixture of solution A (water/acetonitrile/trifluoroacetic acid, 95:5:0.1) and solution B (water/acetonitrile/trifluoroacetic acid, 5:95:0.1). The absorbance was measured at 220 nm.

### CE-HPLC Analysis of Hemoglobin Tetramers

Cation-exchange HPLC analysis was performed using a NexeraX2 SIL-30AC chromatograph and the LC Solution software (Shimadzu). A 2 cation-exchange column (PolyCAT A, PolyLC, Columbia, MD) was used to separate hemoglobin tetramers by HPLC. Samples were eluted with a gradient mixture of solution A (20 mM bis Tris, 2 mM KCN, pH=6.5) and solution B (20 mM bis Tris, 2 mM KCN, 250 mM NaCl, pH=6.8). The absorbance was measured at 415 nm.

### HSPC Xenotransplantation in NBSGW Mice

NOD.Cg-Kit^{W-41J}Tyr⁺Prkdc^{scid}Il2rg^{tmlWjl}/ThomJ (NBSGW) mice were housed in a pathogen-free facility. Control or edited mobilized healthy donor or β-thalassemic CD34⁺ cells (2.6×10⁵ cells per mouse) were transplanted into nonirradiated NBSGW male and female mice of 6 to 9 weeks of age via retro-orbital sinus injection. NBSGW male and female mice were conditioned with busulfan (Sigma, St Louis, MO, US) injected intraperitoneally (15 mg/kg body weight) 24h before transplantation. 16 weeks after transplantation, NBSGW primary recipients were sacrificed. Cells were harvested from bone marrow, thymus, spleen, and blood, 10⁵ cells from each organ were stained with antibodies against murine and human surface markers: murine CD45 (1/50 mCD45-VioBlue, Miltenyi Biotec), human CD45 (1/50 hCD45-APCvio770, Miltenyi Biotec), human CD3 (1/50 CD3-APC, Miltenyi Biotec), human CD14 (1/50 CD14-PECy7, BD Biosciences), human CD15 (1/50 CD15-PE), Miltenyi Biotec; human CD11b (1/100 CD11b-APC, Miltenyi Biotec), human CD19 (1/100 CD19-BV510, BD Biosciences), human CD235a (1/50 CD235a-PE, BD Biosciences), human CD71 (1/10 CD71-APC, BD Biosciences), CD36 (1/50 CD36-FITC, BD Biosciences), CD34 (1/100 CD34-PE-Vio770, Miltenyi Biotec) and analyzed by flow cytometry using the Novocyte analyzer (Agilent) and the FlowJo software (BD Biosciences). Human bone marrow CD45+ cells were sorted by immunomagnetic selection with CD45 MicroBeads (Miltenyi Biotec). All experiments and procedures were performed in compliance with the French Ministry of Agriculture's regulations on animal experiments and were approved by the regional Animal Care and Use Committee (APAFIS #2019061312202425_v4). Mice were housed in a temperature (20-22° C.) and humidity (40-50%)-controlled environment with 12 h/12 h light/dark cycle and fed ad libitum with a standard diet.

### BM Cell Sorting

BM cells were subjected to an immunostaining with biotinylated antibodies recognized the following surface markers: CD3 (dilution 1/25, clone HIT3a, BD), CD19 (dilution 1/25, clone HIB19, BD), B220 (dilution 1/50, clone RA3-6B2, BD), Ter119 (dilution 1/50, clone TER-119, BD), mCD117 (clone 2B8, BD). BM cells were washed and incubated with 20 μL of Anti-Biotin beads (Miltenyi Biotec). After washing, cells were magnetically purified using LS column (Milteniy Biotec) according to manufacturer's instructions. Cells from the negative fraction were immunostained with the following antibodies: CD235a-PE (dilution 1/5000, BD) and hCD45-BV510 (dilution 1/100, BD). The hCD45^{low/-}/CD235a^{high} cells were sorted using the MA900 cell sorter (Sony Biotechnology) and subjected to flow cytometry and RP-HPLC analysis.

### Colony-Forming Cell (CFC) Assay

For in vitro and in vivo experiments, CD34⁺ HSPCs and human bone marrow CD45⁺ cells were plated at a concentration of 500 cells/mL (according to pre-nucleofection counting) or 200.000 cells/mL, respectively, in a methylcellulose-based medium (Stem Cell Technologies, GFH4435) under conditions supporting erythroid and granulo-monocytic differentiation. BFU-E and CFU-GM colonies were counted after 14 days. Single colonies were collected to evaluate base editing efficiency and InDels.

### Macrophage Depletion and Human RBC Sorting

15 weeks after HSPC infusion, mice were injected intraperitoneally with 10 μl/g clodronate liposomes (5 mg/mL; Liposoma). 4 days after treatment, 10 μL of whole blood were stained with antibodies recognizing the following murine and human surface markers: murine CD45 (1/50 mCD45-VioBlue, Miltenyi Biotec), human CD45 (1/50 hCD45-APCvio770, Miltenyi Biotec), CD235a (1/5000, CD235a-PE, BD) and Ter 19 (1/50, Ter119-FITC, BD). After two washings, human RBCs were sorted using MA900 cell sorter (Sony Biotechnology). After gating on the mCD45⁻/hCD45^{low/-} population, a minimum of 50,000 human RBCs (CD235a^{high}/Ter119⁻) were sorted and lysed for RP-HPLC analysis.

### Results

### Adenine Base Editing is an Efficient Tool to Revert the IVS1-110 (G>A) Mutation

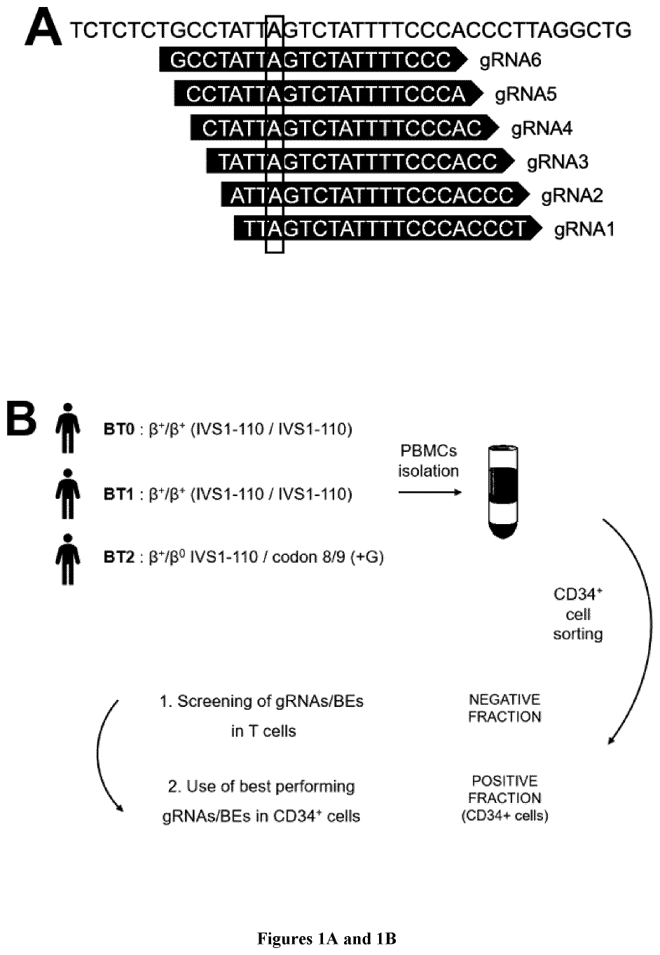

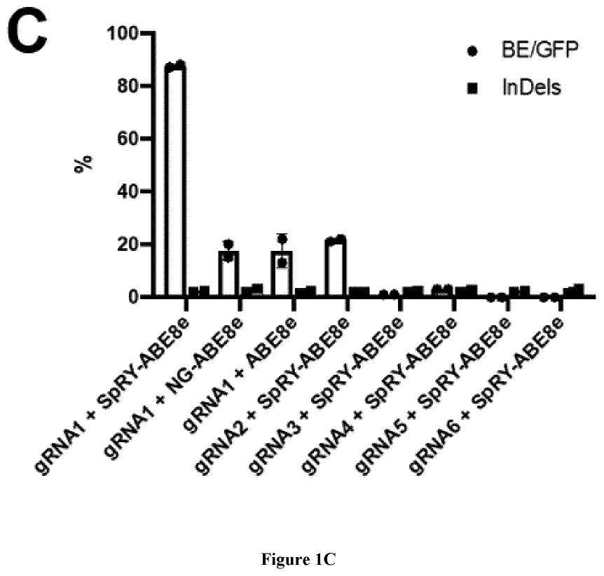

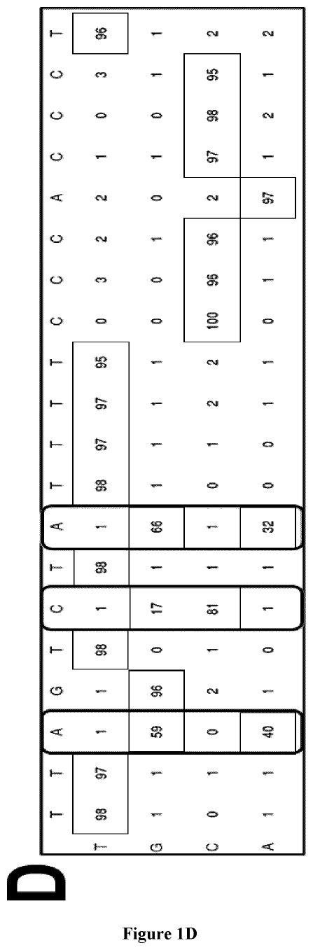

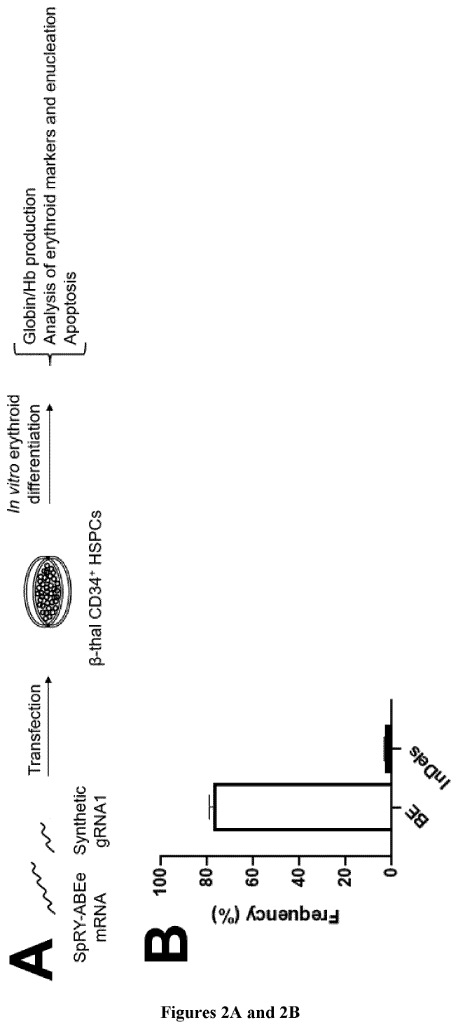

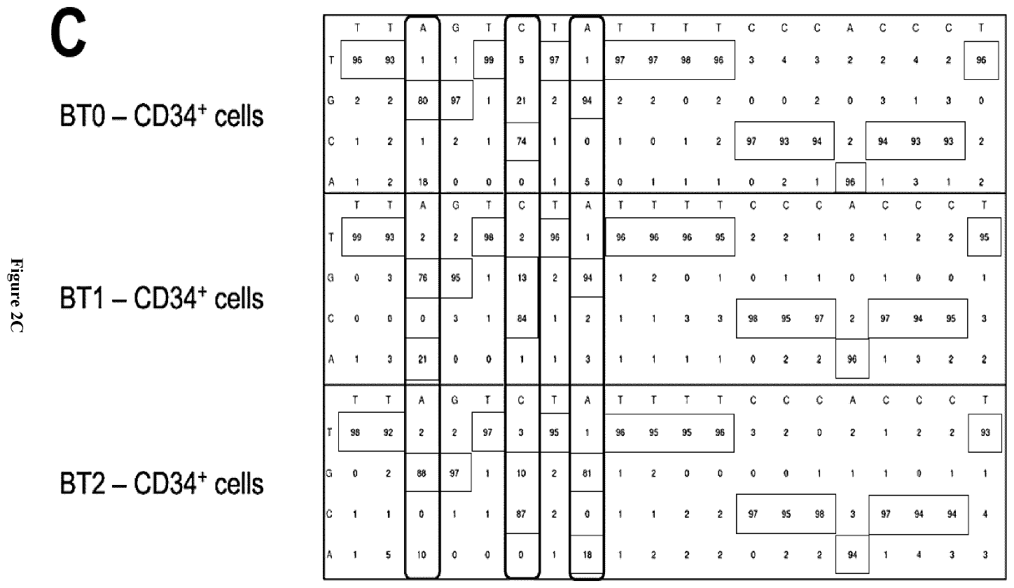

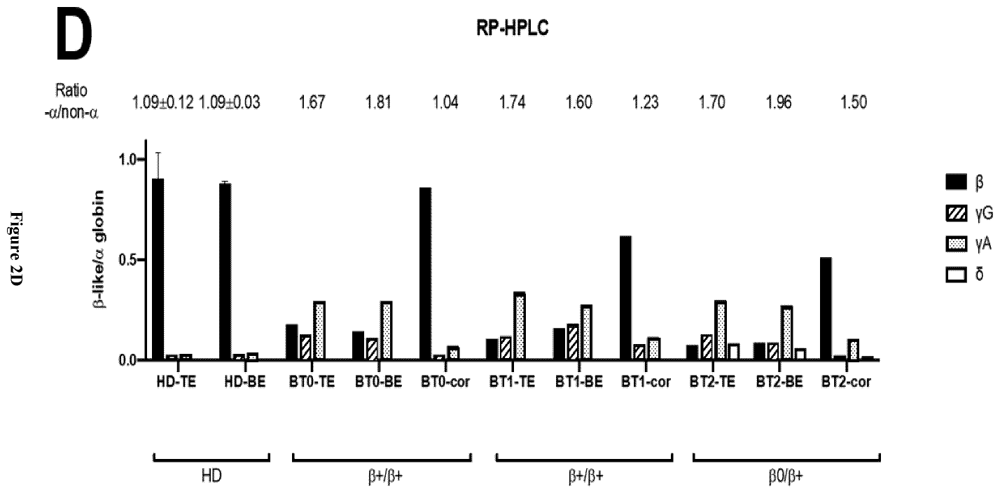

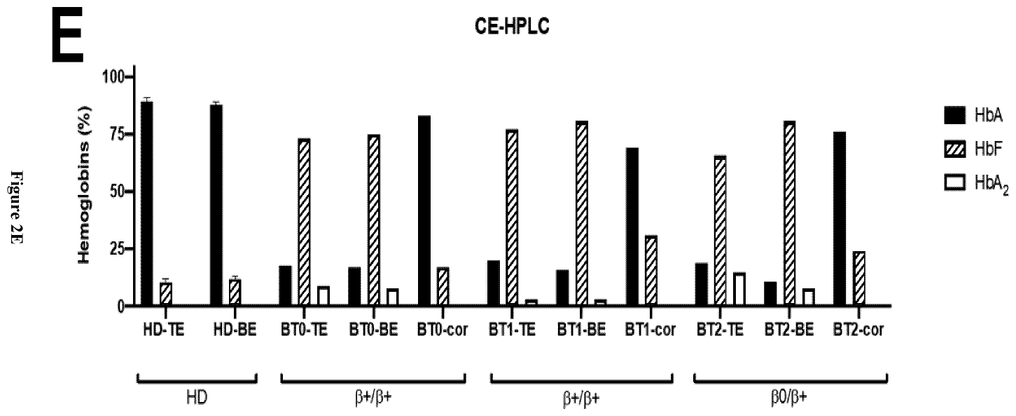

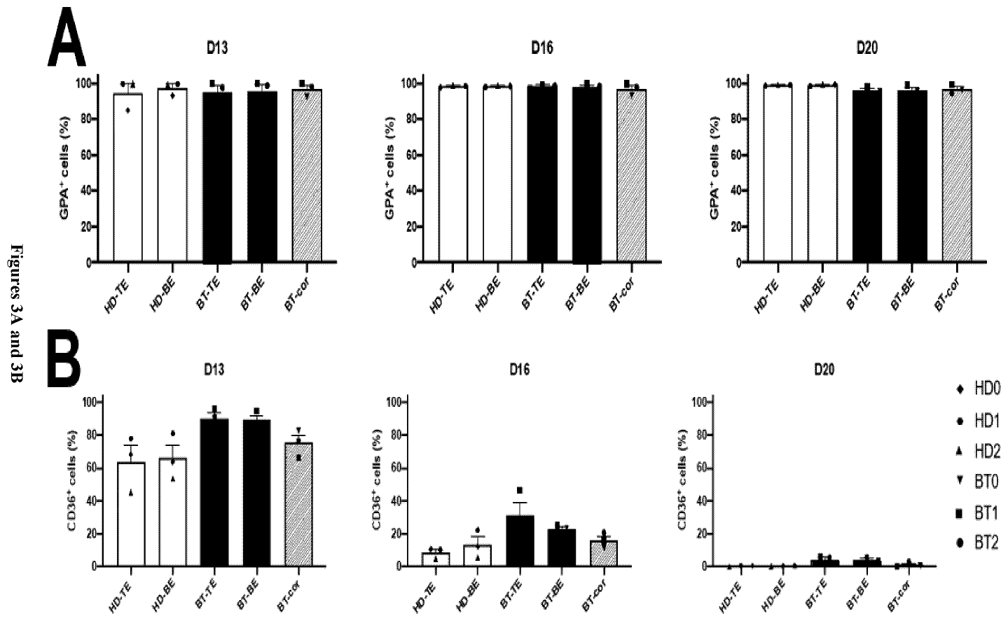

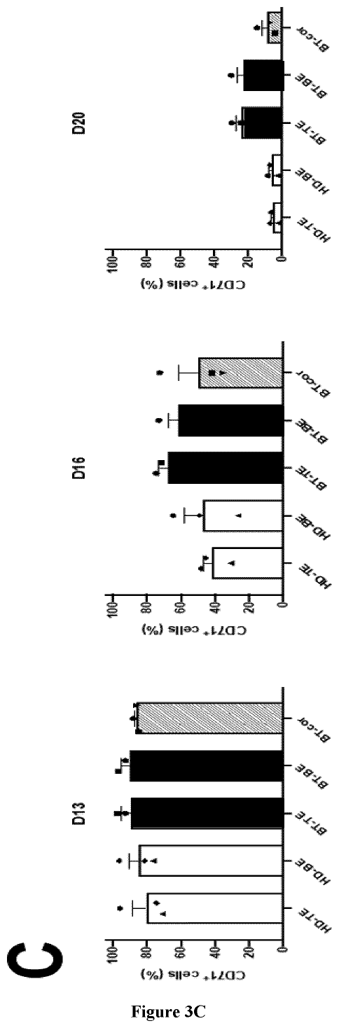

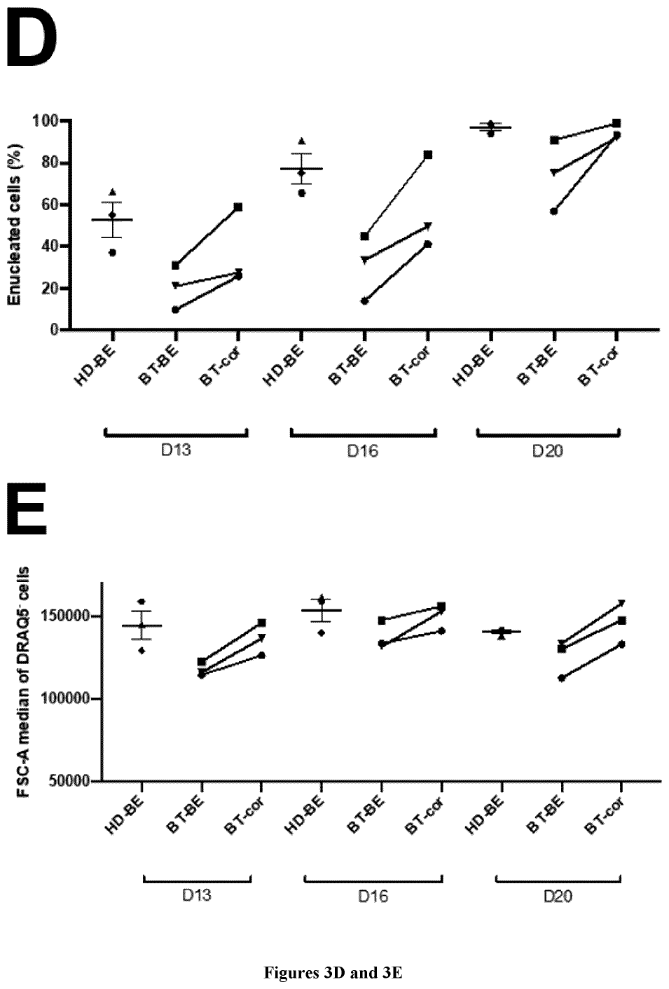

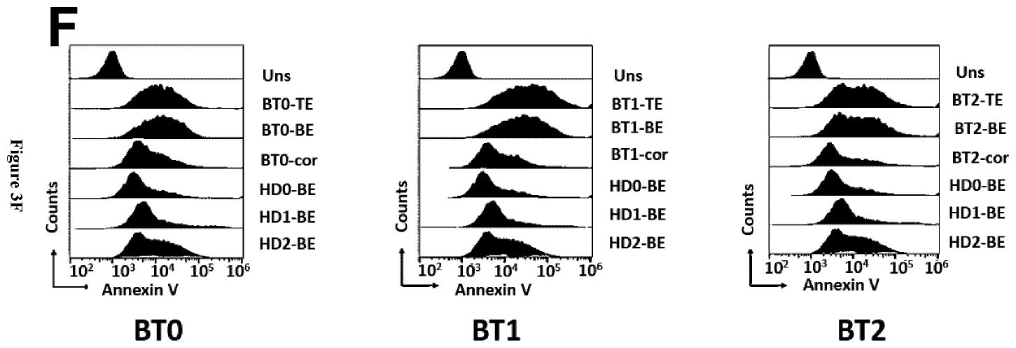

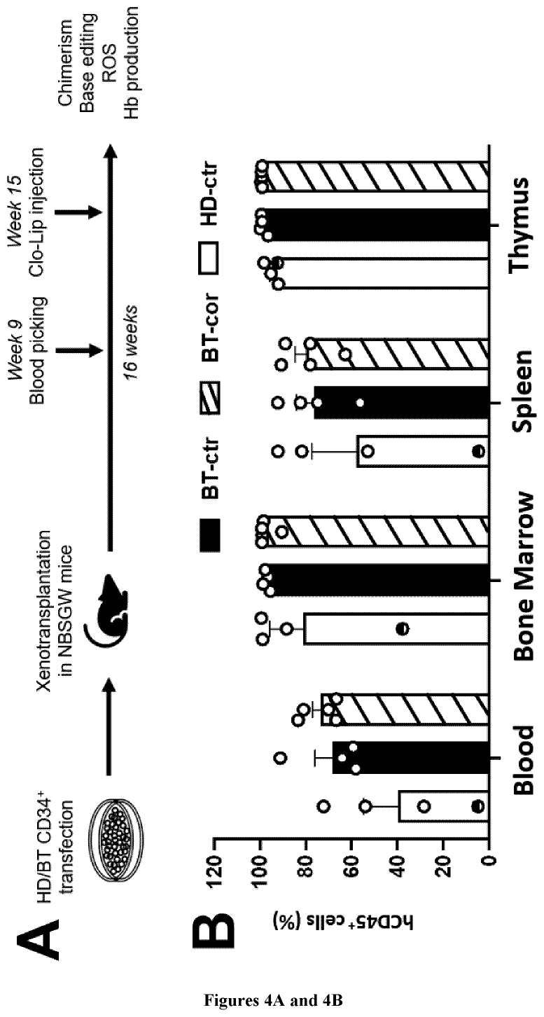

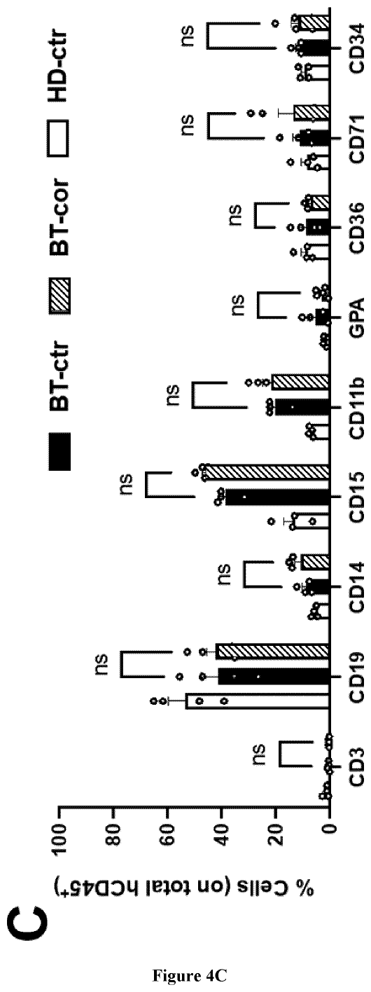

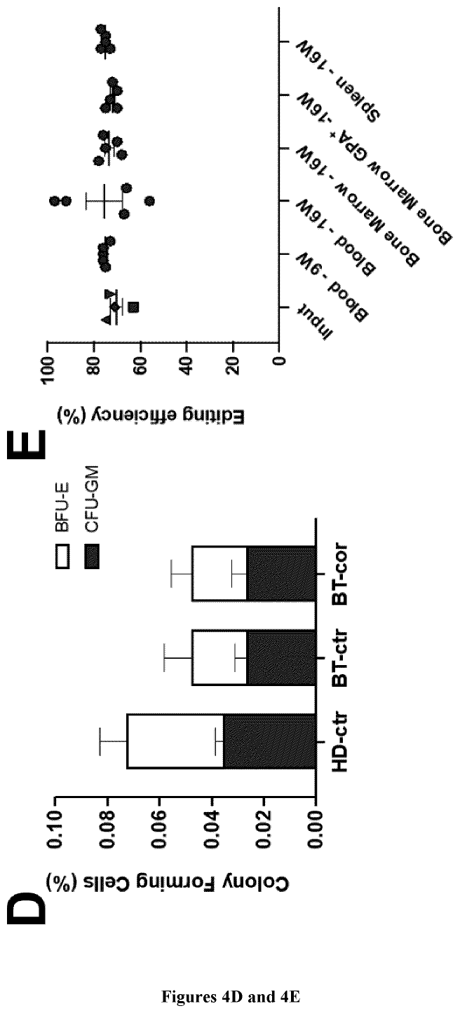

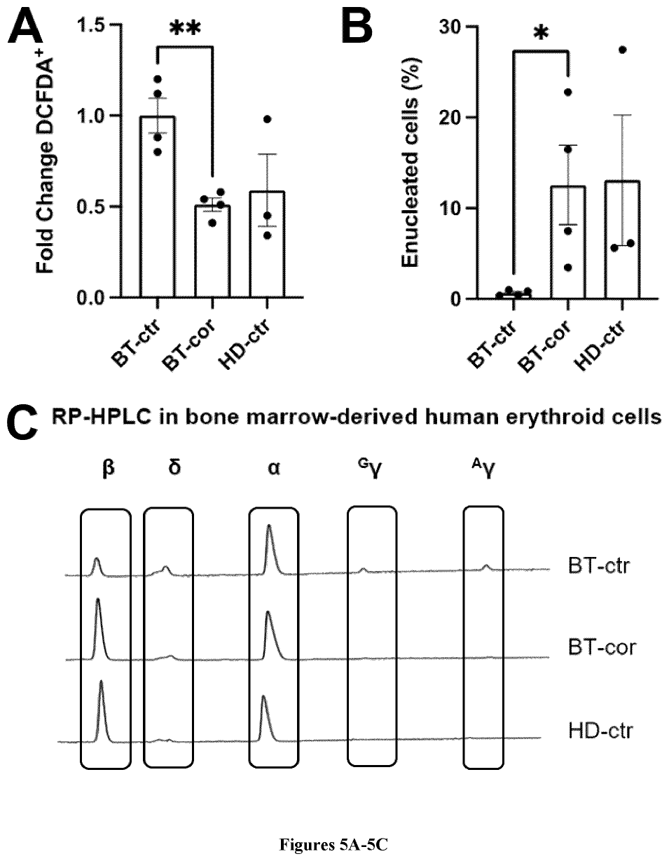

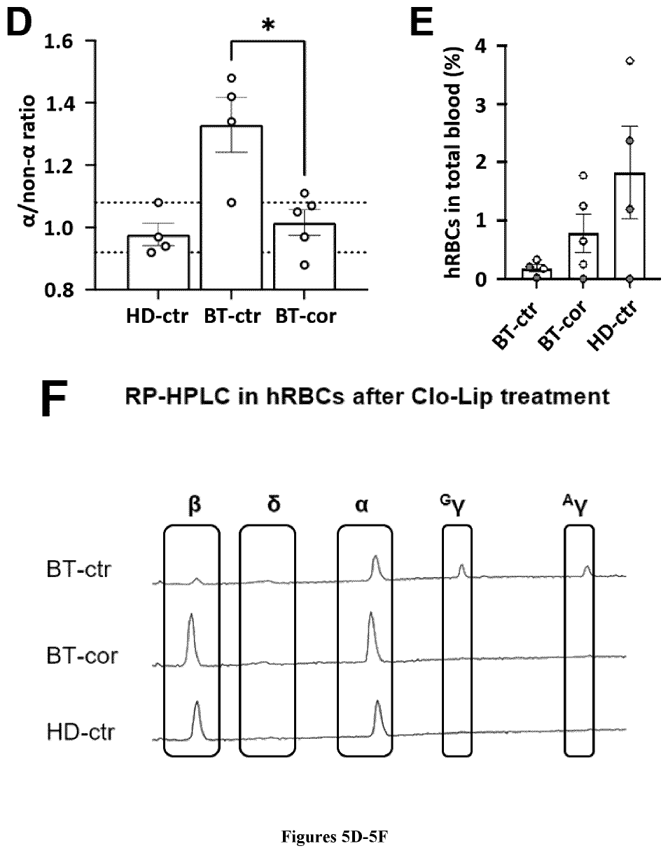

ABEs allow A>G conversions and can potentially correct the IVS1-110 (G>A) mutation. In particular, we used ABEs ABE8e and NG-ABE8e²⁷ recognizing NGG and NG PAM, respectively, and SpRY-ABE8e²⁸, an ABE that we generated by combining the highly processive deaminase from ABE8e²⁷ with the SpRY PAM-less Cas9 nickase²⁹ (SpRY-ABE8e). This latter ABE allowed the design of 6 gRNAs (1 to 6) placing the target base within positions 3 to 8 of the canonical editing window (FIG. 1A). Only gRNA1 was compatible all the 3 ABEs (Table 1).

We screened gRNA/BE combinations in T cells obtained from β-thalassemia patients homozygous for the IVS1-110 (G>A) mutation (FIG. 1B). Cells were transfected with chemically modified gRNAs and in vitro transcribed ABE mRNAs. gRNA1 and SpRY-ABE8e was the most efficient combination able to revert the IVS1-110 (G>A) mutation in nearly 90% of the HBB mutant alleles, as evaluated by Sanger sequencing and EditR analysis (FIGS. 1C-D). Two additional modifications were observed in the target sequence (bystander edits) (FIG. 1D). However, these changes have likely no consequences on HBB expression as they occur in an intronic region and SNPs described at these positions were not associated with any clinical manifestation.

Efficient Correction of the IVS1-110 (G>A) Mutation in Li-Thalassemic HSPCs Restores Normal Hb Production in their Erythroid Progeny In Vitro

HSPCs from 3 different β-thalassemia patients were transfected with chemically modified gRNA1 and in vitro transcribed ABE8e-SpRY mRNA (FIGS. 1B, 2A). 2 donors were homozygous for the IVS1-110 (G>A) mutation (BT0 and BT1) and the third one was a compound heterozygous harboring IVS1-110 (G>A) mutation and the codon 8/9 (+G) β⁰ mutation (FIG. 1B). Correction of the targeted mutation was highly efficient and similar between the 3 donors (77.3% ±1.3) (FIGS. 2B-C). Sanger sequencing and TIDE analysis confirmed the DSB-free nature of ABEs, as we detected no InDels in base edited samples (FIG. 2B). Following transfection, β-thalassemic HSPCs were differentiated towards the erythroid lineage to evaluate globin and hemoglobin production by HPLC (FIG. 2A). In untreated β-thalassemic cells, we observed residual β-globin expression poorly compensated by fetal γ (γ^{A}+γ^{G})-globin expression, as showed by the elevated ratio between α- and non-α-globins (1.67, 1.74 and 1.70 in BT0-TE, BT1-TE and BT2-TE samples, respectively) (FIG. 2D). After treatment, erythroid cells exhibited higher levels of HbA β-globin, along with lower α/non-α ratios (FIGS. 2D-E). Importantly, the ratio between α- and non-α-globins in edited cells from patients homozygous for the IVS1-110 (G>A) mutation was comparable to the value measured in healthy donors' cells and it was substantially ameliorated in the erythroid cells from the compound heterozygous patient (FIG. 2D).

In conclusion, we were able to efficiently revert the IVS1-110 (G>A) mutation in β-thalassemic HSPCs without causing DSBs, and to restore a normal to nearly normal Hb expression profile in HSPC-derived RBCs.

### Efficient Reversion of the IVS1-110 (G>A) Mutation in β-Thalassemic HSPCs Corrects Ineffective Erythropoiesis In Vitro

In β-thalassemia, α- and β-globin chain imbalance causes premature death via apoptosis of erythroid precursors, thus leading to ineffective erythropoiesis, a hallmark of the disease³⁰. The typical delayed erythroid differentiation of β-thalassemic cells was corrected by our treatment, as evaluated by the flow cytometry analysis of different erythroid markers throughout the differentiation. Indeed, the early erythroid markers CD36 and CD71, were properly downregulated at the end of the differentiation in samples derived from edited HSPCs, similarly to healthy donor samples (FIGS. 3A-C). Moreover, in all the samples, we observed an increased enucleation rate (frequency of DRAQ5⁻ cells) along the differentiation compared to controls. At the end of the differentiation, treated samples reached enucleation levels similar to the frequencies observed in healthy donor erythroid populations (FIG. 3D). Furthermore, the size of enucleated cells (typically reduced in β-thalassemic cells in culture) was increased in edited samples (FIG. 3E). Finally, we evaluated the potential of our treatment to rescue the apoptosis in β-thalassemic cells. Measurement of Annexin⁺ cells by flow cytometry showed a reduced apoptotic rate in edited β-thalassemic samples, as compared to the control cells (FIG. 3F).

In conclusion, we demonstrated that reverting the IVS1-110 (G>A) mutation using base editing corrected in vitro the β-thalassemic cell phenotype in terms of erythroid differentiation, enucleation, RBC size and apoptosis.

Efficient Correction of the IVS1-110 (G>A) Mutation in Li-Thalassemic HSPCs Restores Normal Hb Production and Corrects Ineffective Erythropoiesis in their Erythroid Progeny In Vivo

To evaluate the ability of ABE8e-SpRY to correct HBB in repopulating HSCs, we transplanted human β-thalassemic HSPCs homozygous for the IVS1-110 mutation after transfection with ABE8e-SpRY mRNA and gRNA1 into immunodeficient NBSGW mice (FIG. 4A). HD HSPCs and unedited β-thalassemic HSPCs served as controls. Sixteen weeks post-transplantation, no differences were observed between edited and control HSPCs in terms of engraftment and differentiation potential, as measured by the frequency of human CD45⁺ cells in the hematopoietic tissues and the expression of lineage-specific markers (FIGS. 4B-C). Human CD45⁺ bone marrow cells were isolated and subjected to a CFC assay. Control and edited β-thalassemic samples showed a similar number of erythroid and granulo-monocytic colonies, demonstrating no impact of base editing on the clonogenic potential of engrafted human cells (FIG. 4D). Importantly, 16 weeks after transplant on-target base editing efficiency was similar in input HSPCs (70.5% ±2.6) and in bone marrow (73.4% ±1.9), blood (75.6% ±8.0) and spleen (75.4%±0.7), demonstrating a successful engraftment of base edited HSCs (FIG. 4E).

To assess the correction of the β-thalassemic phenotype in the erythroid progeny of HSCs, we sorted human GPA⁺ erythroid cells from the bone marrow (72.0% ±0.9; FIG. 4E). Interestingly, ROS levels and enucleation rate were normalized in erythroid cells derived from edited HSPCs (FIGS. 5A-B). Moreover, HPLC analysis showed the expression of wt β-globin in treated samples and restoration of the α-/non-α-globin ratio (FIGS. 5C-D).

Before sacrifice, mice were subjected to clodronate liposomes (Clo-Lip) treatment in order to evaluate the egression of mature human RBCs from the bone marrow to the blood. Interestingly, we observed and increased frequency of RBCs in mice engrafted with corrected β-thalassemic cells as compared to the levels observed in the control group of mice engrafted with unedited β-thalassemic cells (FIG. 5E). HPLC showed the correction of the β-thalassemic phenotype in circulating hRBCs obtained from the treated group, with a high expression of β-globin and a globin expression profile overlapping with that observed in the HD control group (FIG. 5F).

### REFERENCES

Throughout this application, various references describe the state of the art to which this invention pertains. The disclosures of these references are hereby incorporated by reference into the present disclosure.
1. Modell, B. & Darlison, M. Global epidemiology of haemoglobin disorders and derived service indicators. Bull. World Health Organ. 86, 480-487 (2008).
2. Taher, A. T., Weatherall, D. J. & Cappellini, M. D. Thalassaemia. Lancet Lond. Engl. 391, 155-167 (2018).
3. Thein, S. L. The Molecular Basis of β-Thalassemia. Cold Spring Harb. Perspect. Med. 3, a011700 (2013).
4. Cao, A., Gossens, M. & Pirastu, M. Beta thalassaemia mutations in Mediterranean populations. Br. J. Haematol. 71, 309-312 (1989).
5. IthaGenes, IthaID: 113. https://www.ithanet.eu/db/ithagenes (2022).
6. Busslinger, M., Moschonas, N. & Flavell, R. A. Beta+thalassemia: aberrant splicing results from a single point mutation in an intron. Cell 27, 289-298 (1981).
7. Sii-Felice, K., Giorgi, M., Leboulch, P. & Payen, E. Hemoglobin disorders: lentiviral gene therapy in the starting blocks to enter clinical practice. Exp. Hematol. 64, 12-32 (2018).
8. Cavazzana, M., Antoniani, C. & Miccio, A. Gene Therapy for P-Hemoglobinopathies. Mol. Ther. 25, 1142-1154 (2017).
9. Park, S. H. et al. Highly efficient editing of the β-globin gene in patient-derived hematopoietic stem and progenitor cells to treat sickle cell disease. Nucleic Acids Res. 47, 7955-7972 (2019).
10. Xu, S. et al. Editing aberrant splice sites efficiently restores β-globin expression in (3-thalassemia. Blood 133, 2255-2262 (2019).
11. Patsali, P. et al. Correction of IVS I-110(G>A) 0-thalassemia by CRISPR/Cas- and TALEN-mediated disruption of aberrant regulatory elements in human hematopoietic stem and progenitor cells. Haematologica 104, e497-e501 (2019).
12. Milyavsky, M. et al. A Distinctive DNA Damage Response in Human Hematopoietic Stem Cells Reveals an Apoptosis-Independent Role for p53 in Self-Renewal. Cell Stem Cell 7, 186-197 (2010).
13. Cromer, M. K. et al. Global Transcriptional Response to CRISPR/Cas9-AAV6-Based Genome Editing in CD34+ Hematopoietic Stem and Progenitor Cells. Mol. Ther. J. Am. Soc. Gene Ther. 26, 2431-2442 (2018).
14. Schiroli, G. et al. Precise Gene Editing Preserves Hematopoietic Stem Cell Function following Transient p53-Mediated DNA Damage Response. Cell Stem Cell 24, 551-565.e8 (2019).
15. Haapaniemi, E., Botla, S., Persson, J., Schmierer, B. & Taipale, J. CRISPR-Cas9 genome editing induces a p53-mediated DNA damage response. Nat. Med. 24, 927-930 (2018).
16. Kosicki, M., Tomberg, K. & Bradley, A. Repair of double-strand breaks induced by CRISPR-Cas9 leads to large deletions and complex rearrangements. Nat. Biotechnol. 36, 765-771 (2018).
17. Boutin, J. et al. CRISPR-Cas9 globin editing can induce megabase-scale copy-neutral losses of heterozygosity in hematopoietic cells. Nat. Commun. 12, 4922 (2021).
18. Leibowitz, M. L. et al. Chromothripsis as an on-target consequence of CRISPR-Cas9 genome editing. Nat. Genet. 53, 895-905 (2021).
19. Turchiano, G. et al. Quantitative evaluation of chromosomal rearrangements in gene-edited human stem cells by CAST-Seq. Cell Stem Cell 28, 1136-1147.e5 (2021).
20. Zeng, J. et al. Therapeutic base editing of human hematopoietic stem cells. Nat. Med. 26, 535-541 (2020).
21. Rees, H. A. & Liu, D. R. Base editing: precision chemistry on the genome and transcriptome of living cells. Nat. Rev. Genet. 19, 770-788 (2018).
22. Antoniou, P., Miccio, A. & Brusson, M. Base and Prime Editing Technologies for Blood Disorders. Front. Genome Ed. 3, 618406 (2021).
23. Giarratana, M.-C. et al. Ex vivo generation of fully mature human red blood cells from hematopoietic stem cells. Nat. Biotechnol. 23, 69-74 (2005).
24. Weber, L. et al. Editing a 7-globin repressor binding site restores fetal hemoglobin synthesis and corrects the sickle cell disease phenotype. Sci. Adv. 6, (2020).
25. Kluesner, M. G. et al. EditR: A Method to Quantify Base Editing from Sanger Sequencing. CRISPR J. 1, 239-250 (2018).
26. Brinkman, E. K., Chen, T., Amendola, M. & van Steensel, B. Easy quantitative assessment of genome editing by sequence trace decomposition. Nucleic Acids Res. 42, e168 (2014).
27. Richter, M. F. et al. Phage-assisted evolution of an adenine base editor with improved Cas domain compatibility and activity. Nat. Biotechnol. 38, 883-891 (2020).
28. Ren, Q. et al. PAM-less plant genome editing using a CRISPR-SpRY toolbox. Nat. Plants 7, 25-33 (2021).
29. Walton, R. T., Christie, K. A., Whittaker, M. N. & Kleinstiver, B. P. Unconstrained genome targeting with near-PAMless engineered CRISPR-Cas9 variants. Science 368, 290-296 (2020).
30. Musallam, K. M., Rivella, S., Vichinsky, E. & Rachmilewitz, E. A. Non-transfusion-dependent thalassemias. Haematologica 98, 833-844 (2013).

1. A method of restoring normal expression of β-globin in a eukaryotic cell carrying the IVS1-110 (G>A) mutation comprising the step of contacting the eukaryotic cell with a gene editing platform a comprising (a) at least one adenine base-editor (ABE) and (b) at least one guide RNA molecule for guiding the adenine base-editor to at least one target sequence comprising the IVS1-110 (G>A) mutation, thereby restoring the normal expression of β-globin in the eukaryotic cell.

2. The method of claim 1 wherein the eukaryotic cell is selected from the group consisting of hematopoietic progenitor cells, hematopoietic stem cells (HSCs), pluripotent cells and induced pluripotent stem cells (iPS).

3. The method of claim 1 wherein the eukaryotic cell is homozygous or heterozygous for the IVS1-110 (G>A) mutation.

4. The method of claim 1 wherein the at least one adenine base-editor comprises a defective CRISPR/Cas nuclease.

5. The method of claim 4 wherein the defective CRISPR/Cas nuclease is a nickase.

6. The method of claim 5 wherein the nickase comprises the amino acid sequence as set forth in SEQ ID NO: 2 or SEQ ID NO:3.

7. The method of claim 1 wherein the at least one adenine base-editor comprises a non-nuclease DNA modifying enzyme that is an adenosine deaminase.

8. The method of claim 1 wherein the at least one adenine base-editor consists of the amino acid sequence as set forth in SEQ ID NO:8 (SpRY-ABE8e).

9. The method of claim 1 wherein the at least one guide RNA molecule targets a sequence selected from Table 1.

10. The method of claim 1 wherein the at least one adenine base-editor is an adenine base editor SpRY-ABE8e and b) and the at least one guide RNA molecule is a guide RNA molecule that targets a sequence selected from Table 1.

11. The method of claim 1 wherein the gene editing platform is provided to the eukaryotic cell via ribonucleoprotein (RNP) complexes.

12. The method of claim 1 wherein the gene editing platform is provided to the eukaryotic cell via an RNA-encoded system.

13. A method of treating β-thalassemia in a subject in need thereof, the method comprising transplanting into the subject a therapeutically effective amount of a population of eukaryotic cells obtained by the method of claim 1.

14. The method of claim 13 wherein the population of eukaryotic cells is autologous to the subject.

15. The method of claim 13 wherein the patient suffers from sickle β-thalassemia.

16. The method of claim 5, wherein the nickase is a Cas9 nickase.

17. The method of claim 16, wherein the Cas9 nikase is from S. pyogenes and has a D10A or H840A mutation.
